(19) United States
(12) Patent Application Publication
(10) Pub. No.: US 2025/0044288 A1
(43) Pub. Date: Feb. 6, 2025

(54) CELLULAR ASSAYS AND METHODS TO ASSESS MHC-PEPTIDE-TCR INTERACTIONS AND KINETICS

(71) Applicant: GENENTECH, INC., South San Francisco, CA (US)

(72) Inventors: Darya Yuryevna Orlova, South San Francisco, CA (US); Mayra Cruz Tleugabulova, South San Francisco, CA (US); Martine Abraham Darwish, South San Francisco, CA (US); Jeanne Cheung, South San Francisco, CA (US)

(21) Appl. No.: 18/717,336

(22) PCT Filed: Dec. 7, 2022
(86) PCT No.: PCT/US2022/081066

§ 371 Date: Jun. 6, 2024

Related U.S. Application Data
(60) Provisional application No. 63/287,012, filed on Dec. 7, 2021.
(60) Provisional application No. 63/397,325, filed on Aug. 11, 2022.

(51) Int. Cl. G01N 33/557; G01N 33/569; G01N 33/58

(52) U.S. Cl. CPC G01N 33/557; G01N 33/56977; G01N 33/582; G01N 2333/7051; G01N 2333/70539

(57) ABSTRACT

Compositions, systems and methods for quantifying binding rate kinetics for receptor molecules and MHC molecules are provided. The quantitative data produced can be of an accuracy and quantity suitable for generating predictive models. More specifically, assays described herein can accommodate analysis of large varieties of MHC associated peptides.

### CROSS-REFERENCE TO RELATED APPLICATIONS

This application claims priority benefit of the filing date of U.S. Provisional Patent Application No. 63/287,012, filed on Dec. 7, 2021, the disclosure of which is incorporated by reference herein in its entirety. This application also claims priority benefit of the filing date of U.S. Provisional Patent Application No. 63/397,325, filed on Aug. 11, 2022, the disclosure of which is incorporated by reference herein in its entirety.

### FIELD OF THE INVENTION

This description is generally directed towards methods and assays for assessing major histocompatibility complex displaying peptide (pMHC) and T cell receptor interactions and kinetics. More specifically, the description is directed toward assessing binding kinetics for pMHC and receptor in a context of selection and activation using cell-based assays.

### BACKGROUND OF THE INVENTION

Accurately predicting conditions under which an adaptive immune response is activated continues to elude scientists and medical practitioners. When applying immunotherapy techniques, no existing technologies can accurately predict how a subject will respond. For example, only 20%-40% of patients respond to immunotherapy. Additionally, existing drugs can sometimes activate a broad range of immune cells and sometimes trigger auto-immune reactions. Sharma P, Hu-Lieskovan S, Wargo J A, Ribas A. Primary, Adaptive, and Acquired Resistance to Cancer Immunotherapy. Cell. 2017 Feb. 9; 168(4):707-723. doi: 10.1016/j.cell.2017.01.017. PMID: 28187290; PMCID: PMC5391692.

An understanding of the interactions and kinetics between antigen-presenting cells (APCs) and T cells is limited because a variety of surface molecules of the APCs interact with a variety of surface molecules of the T cells. Recently, it has also become apparent that some of these interactions may not be replicated in in vitro or non-living systems. For example, surface plasmon resonance-based assays fail to consider the contributing factors on binding kinetics associated with live cells. The tetramer stain is the current gold standard for assessing specificity of T cells but has no way to collect comparable rate constant data relating to binding kinetics. Additionally, predictive models need large amounts of data, and such techniques require a prohibitively substantial time investment.

As such, there is a need for cell-based assays for measuring kinetics between antigen-presenting cells (APCs) and T cells quickly and in a way that generates comparable data at a low cost. Such assays will also lead to higher response rates for patients while, at the same time, reducing the severity of or eliminating auto-immune reactions. The present disclosure addresses these and other needs.

### SUMMARY OF THE INVENTION

Aspects of the invention include compositions for use in a biokinetic assays, comprising: a polypeptide molecule comprising an antigen-binding region; one or more fluorophores linked to the polypeptide molecule; and an antigenic peptide bound to the antigen-binding region of the polypeptide molecule.

Aspects of the invention include compositions for use in a biokinetic assay, comprising: a polypeptide molecule comprising an antigen-binding region; one or more fluorophores linked to the polypeptide molecule; and an antigenic peptide bound to the antigen-binding region of the polypeptide molecule, wherein the antigenic peptide comprises a UV cleavable amino acid.

In some embodiments, a polypeptide molecule comprises a single polypeptide chain. In some embodiments, the polypeptide molecule comprises a first polypeptide chain and a second polypeptide chain.

In some embodiments, an antigen-binding region comprises at least a portion of the first polypeptide chain of the polypeptide molecule. In some embodiments, the first polypeptide chain comprises an α₁ domain and an α₂ domain. In some embodiments, the first polypeptide chain comprises an α₃ domain. In some embodiments, the first polypeptide chain comprises an amino acid sequence of a recombinant human leukocyte antigen. In some embodiments, the first polypeptide chain comprises an amino acid sequence of a recombinant murine histocompatibility system 2. In some embodiments, the second polypeptide chain is linked to the α₃ domain of the first polypeptide chain. In some embodiments, the second polypeptide chain comprises an amino acid sequence of a β₂-microglobulin molecule.

In some embodiments, an antigen-binding region comprises at least a portion of the first polypeptide chain and at least a portion of the second polypeptide chain. In some embodiments, the portion of the first polypeptide chain comprises an a domain, the portion of the second polypeptide chain comprises a β₁ domain, and the α₁ domain and the β₁ domain form the antigen-binding region. In some embodiments, the first polypeptide chain further comprises an α₂ domain. In some embodiments, the second polypeptide chain further comprises a β₂ domain. In some embodiments, the first polypeptide chain comprises an amino acid sequence of a recombinant human leukocyte antigen. In some embodiments, the second polypeptide chain comprises a polypeptide sequence of a recombinant human leukocyte antigen. In some embodiments, the antigen-binding region comprises at least one α-helix. In some embodiments, the antigen-binding region comprises at least one β-sheet.

In some embodiments, the one or more fluorophores are covalently linked to the polypeptide molecule. In some embodiments, the covalent linkage comprises an ester. In some embodiments, the one or more fluorophores are covalently linked to one or more solvent exposed surface lysine residues of the polypeptide molecule.

In some embodiments, the first and second polypeptide chains are non-covalently linked. In alternative and additional embodiments, the first and second polypeptide chains are covalently linked.

In some embodiments, the polypeptide molecule is bound to an antigen presenting cell surrogate. In some embodiments, the antigen presenting cell surrogate comprises a bead. In some embodiments, the polypeptide molecule is attached to the antigen presenting cell surrogate by a linker. In some embodiments, the linker comprises a polyethylene glycol (PEG) molecule.

In some embodiments, the composition comprises a receptor bound to the antigen-binding region of the polypeptide molecule.

In some embodiments, the composition comprises a live lymphocyte. In some embodiments, at least a portion of the receptor is on a cell membrane of the live lymphocyte. In some embodiments, the composition comprises a co-receptor on the cell membrane of the live lymphocyte. In some embodiments, the co-receptor is bound to a portion of the polypeptide molecule. In some embodiments, the live lymphocyte is a T cell and the receptor is a T cell receptor (TCR). In some embodiments, the co-receptor comprises a CD8 molecule. In some embodiments, the live lymphocyte is a B cell and the receptor is a B cell receptor (BCR). In some embodiments, the co-receptor comprises a CD4 molecule. In some embodiments, the live cell comprises a macrophage and the receptor comprises a chemokine receptor. In some embodiments, the live cell comprises a dendritic cell and the receptor comprises a pattern recognition receptor (PRR).

In some embodiments, the antigenic peptide has a length that ranges from 8-11 amino acid residues. In other embodiments, the antigenic peptide has a length that ranges from 15-24 residues. In some embodiments, the antigenic peptide comprises a UV cleavable moiety.

Aspects of the invention include a reaction mixture for generating a probe complex for use in a biokinetic assay, the mixture comprising any of the compositions above and herein and a target antigenic peptide.

In some embodiments, the target antigenic peptide is present at a concentration in a molar excess compared to the antigenic peptide bound to the antigen-binding region of the polypeptide molecule. In some embodiments, the concentration of the target antigenic peptide is 25 times greater than the concentration of the antigenic peptide.

In some embodiments, the reaction mixture comprises 25 mM TRIS. In some embodiments, the reaction mixture is at a pH of 8.0. In some embodiments, the reaction mixture comprises 150 mM NaCl. In some embodiments, the reaction mixture comprises 4 mM EDTA. In some embodiments, the reaction mixture comprises 5% ethylene glycol.

Aspects of the invention include methods of generating a monomeric probe complex, comprising: contacting a polypeptide molecule comprising an antigen binding region with a first antigenic peptide to generate an antigen-presenting complex; contacting the antigen-presenting complex with a plurality of fluorophore molecules to generate a fluorophore-labeled antigen-presenting complex; determining a quantity of fluorophore molecules covalently bound to the fluorophore-labeled antigen-presenting complex; and exchanging the first antigenic peptide with a second antigenic peptide to generate the monomeric probe complex.

In some embodiments, the methods of generating a monomeric probe complex comprise exchanging the first antigenic peptide with the second antigenic peptide comprises cleaving the first antigenic peptide to generate a cleaved first antigenic peptide. In some embodiments, the cleaved first antigenic peptide has a lower binding affinity to the antigen binding region than the first antigenic peptide. In some embodiments, the second antigenic peptide has a higher affinity for the antigen binding region than the cleaved first antigenic peptide.

In some embodiments, the methods of generating a monomeric probe complex comprise cleaving the first antigenic peptide comprises applying UV radiation. In some embodiments, the UV radiation comprises a wavelength of 365 nanometers.

In some embodiments, the step of contacting the antigen-presenting complex with a plurality of fluorophore molecules to generate a fluorophore-labeled antigen-presenting complex comprises covalently linking one or more solvent exposed surface lysine residues of the polypeptide molecule to one or more of the fluorophore molecules.

In some embodiments, the methods of generating a monomeric probe complex comprise separating one or more unconjugated fluorophores from the labeled monomeric probe complex. In some embodiments, the methods of generating a monomeric probe complex comprise determining a concentration of a plurality of labeled monomeric probe complexes.

In some embodiments, the methods of generating a monomeric probe complex comprise determining an average number of fluorophores conjugated to each of a plurality of the labeled monomeric probe complexes. In some embodiments, the step of determining the average number of fluorophores conjugated to each of the plurality of the labeled monomeric probe complexes includes using a plurality of relative abundance values, wherein each relative abundance value corresponds to a different number of conjugated fluorophores. In some embodiments, each of the plurality of relative abundance values are determined using mass spectrometry.

Aspects of the invention include methods for collecting association rate data for assessing live cell activation, comprising: contacting a plurality of live cells with a plurality of compositions of any one of the compositions described herein and above at a concentration, wherein each of the plurality of live cells comprises a plurality receptor molecules on a cell membrane; binding the receptor molecules to the compositions over a time interval to form a plurality of receptor-probe complexes, wherein each receptor-probe complex comprises one composition bound to one receptor; collecting at least two samples of the plurality of live cells at different time points over the time interval; contacting the live cells in each of the at least two samples with a fixing agent to preserve the receptor-probe complexes and to prevent further binding between the receptor molecules and the compositions; determining a number of receptor-probe complexes on the cell membrane of each cell; and analyzing the number of receptor-probe complexes on the cell membrane of each cell in each sample to collect the association rate data.

In some embodiments, the live cells comprise a T cell. In some embodiments, the live cells comprise a B cell. In some embodiments, the live cells comprise a macrophage. In some embodiments, the live cells comprise a dendritic cell.

In some embodiments, the signal intensity is measured using an analytical device. In some embodiments, the analytical device comprises a flow cytometer. In some embodiments, the analytical device comprises a fluorometer.

In some embodiments, the fixation agent comprises paraformaldehyde. In some embodiments the methods comprise preventing receptor internalization by decreasing a temperature of the receptor-probe complexes. In some embodiments, the temperature is decreased to 4° C. In some embodiments the methods comprise separating one or more unbound compositions from the plurality of live cells.

In some embodiments, each of the receptor-probe complexes comprises a CD8 molecule. In some embodiments, each of the receptor-probe complexes comprises a CD4 molecule.

Aspects of the invention include methods for collecting dissociation rate data for assessing live cell activation, comprising: contacting a plurality of live cells with a plurality of any of the compositions described herein and above at a concentration, wherein each of the plurality of live cells comprises a plurality receptor molecules on a cell membrane; binding the receptor molecules to the compositions to form a plurality of receptor-probe complexes until an equilibrium is achieve, wherein each receptor-probe complex comprises one composition bound to one receptor; dissociating a portion of the receptor-probe complexes over a time interval by reducing the concentration of the compositions; collecting at least two samples of the plurality of live cells at different time points over the time interval; contacting the live cells in each of the at least two samples with a fixing agent to preserve the receptor-probe complexes and to prevent further binding between the receptor molecules and the compositions; determining a number of receptor-probe complexes on the cell membrane of each cell; and analyzing the number of receptor-probe complexes on the cell membrane of each cell in each sample to collect the association rate data.

In some embodiments, the live cells comprise a T cell. In some embodiments, the live cells comprise a B cell. In some embodiments, the live cells comprise a macrophage. In some embodiments, the live cells comprise a dendritic cell. In some embodiments, the signal intensity is measured using an analytical device. In some embodiments, the analytical device comprises a flow cytometer. In some embodiments, the analytical device comprises a fluorometer. In some embodiments, the fixation agent comprises paraformaldehyde.

In some embodiments the methods comprise preventing receptor internalization by decreasing a temperature of the receptor-probe complexes. In some embodiments, the temperature is decreased to 4° C.

In some embodiments the methods comprise separating one or more unbound compositions from the plurality of live cells. In some embodiments, each of the receptor-probe complexes comprises a CD8 molecule. In some embodiments, each of the receptor-probe complexes comprises a CD4 molecule.

In some embodiments, the method further comprises permeabilizing the cell membrane of each cell. In some embodiments, the method further comprises applying a detection reagent for detecting a level of phosphorylation of an intracellular domain of each of the live cells. In some embodiments, the intracellular domain comprises a ζdomain. In some embodiments, the detection reagent comprises an antibody.

Aspects of the invention include systems for measuring binding kinetics for assessing live cell activation, comprising: an analytical device comprising an analysis chamber. In some embodiments, the analysis chamber comprises a composition described above and herein. In some embodiments, the analysis device comprises: an optical source for interrogating the analysis chamber; and a detector for detecting a signal.

In some embodiments, the system further comprises a computer system comprising a non-transitory computer readable storage medium, wherein the non-transitory computer readable storage medium comprises instructions for analyzing the signal. In some embodiments, analyzing the signal comprises normalizing the signal to a previously determining calibration value.

In some embodiments, the analysis chamber comprises a flow cell. In some embodiments, the analytical device comprises a flow cytometer. In some embodiments, the analytical device comprises a fluorometer. In some embodiments, the analytical device comprises a microscope. In some embodiments, the analytical device comprises a mass spectrometer.

Aspects of the invention include systems for measuring binding kinetics for assessing live cell activation, comprising: an analytical device comprising an analysis chamber. In some embodiments, the analysis chamber comprises a reaction mixture described above and herein.

In some embodiments, the analysis device comprises: an optical source for interrogating the analysis chamber; and a detector for detecting a signal.

In some embodiments, the system further comprises a computer system comprising a non-transitory computer readable storage medium, wherein the non-transitory computer readable storage medium comprises instructions for analyzing the signal. In some embodiments, analyzing the signal comprises normalizing the signal to a previously determining calibration value.

In some embodiments, the analysis chamber comprises a flow cell. In some embodiments, the analytical device comprises a flow cytometer. In some embodiments, the analytical device comprises a fluorometer. In some embodiments, the analytical device comprises a microscope. In some embodiments, the analytical device comprises a mass spectrometer.

In one aspect, a method for collecting biophysical parameter data for assessing a T cell receptor (TCR) and a peptide-major histocompatibility complex (MHC) association rate constant is described according to various embodiments. In various embodiments, the method comprises generating a set of monomeric probes. In various embodiments, each monomeric probe comprises a detection molecule and one MHC comprising a peptide. In various embodiments, the method comprises associating TCR molecules with the monomeric probes in a one-to-one correspondence to form TCR-monomeric-probe complexes over a time interval. In various embodiments, two or more subsets of TCR-monomeric-probe complexes over the time interval. In various embodiments, each subset is taken at a different timepoint. In various embodiments, the method comprises preventing formation of new TCR-monomeric-probe complexes within each subset at their corresponding timepoints. In various embodiments, the method comprises measuring a signal intensity from the detection molecules in each subset using an analytical device.

In one aspect, a method for collecting biophysical parameter data for assessing a T cell receptor (TCR) and a peptide-major histocompatibility complex (pMHC) dissociation rate constant is described in accordance with various embodiments. In various embodiments, the method comprises generating a set of monomeric probes, wherein each monomeric probe comprises a detection molecule and one MHC comprising a peptide. In various embodiments, the method comprises associating TCR molecules with the monomeric probes in a one-to-one correspondence to form TCR-monomeric-probe complexes. In various embodiments, the method comprises dissociating the TCR-monomeric-probe complexes into monomeric probes and TCRs over a time interval. In various embodiments, the method comprises sampling two or more subsets of TCR-monomeric-probe complexes over the time interval. In various embodiments, each subset is taken at a different timepoint. In various embodiments, the method comprises preventing dissociation of additional TCR-monomeric-probe complexes within each subset at their corresponding timepoints. In various embodiments, the method comprises measuring a signal intensity from the detection molecules in each subset using a high-throughput analytical device.

These and further aspects will be further explained in the rest of the disclosure, including the Examples.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF FIGURES

The features of the technology are set forth with particularity in the appended claims. A better understanding of the features and advantages of the present technology will be obtained by reference to the following detailed description that sets forth illustrative embodiments, in which the principles of the technology are utilized, and the accompanying drawings (also “Figure” and “FIG.” herein). The accompanying drawings are not intended to be drawn to scale. Like reference numbers and designations in the various drawings indicate like elements. For purposes of clarity, not every component can be labeled in every drawing. In the drawings:

FIG. 1 graphically illustrates T cell selection occurring in the Thymus in accordance with various embodiments.

FIG. 2 is a schematic illustration of a polypeptide molecule of an antigen presenting cell binding to a cell surface protein of a T cell in accordance with various embodiments.

FIG. 3 is a graph showing association signal intensity for different timepoints over a time interval from a cell-based assay for determining an association or dissociation rate in accordance with various embodiments.

FIG. 4 is a schematic illustration showing an example of a lymphocyte and a monomeric probe in accordance with various embodiments.

FIG. 5A is a flow chart of a process for collecting association rate data for assessing live cell activation in accordance with various embodiments.

FIG. 5B is a flow chart of a process for an association assay in accordance with various embodiments.

FIG. 6A is a flow chart of a process for collecting dissociation rate data for assessing live cell activation in accordance with various embodiments.

FIG. 6B is a flow chart of a process for a dissociation assay in accordance with various embodiments.

FIG. 7A is example data in graphical format from an association assay in accordance with various embodiments.

FIG. 7B is example data in graphical format from an association assay in accordance with various embodiments.

FIG. 8A is example data in graphical format from a dissociation assay in accordance with various embodiments.

FIG. 8B is example data in graphical format from a dissociation assay in accordance with various embodiments.

FIG. 9A is an illustration depicting a T Cell Receptor complex comprising multiple proteins positioned within a cell membrane in accordance with various embodiments.

FIG. 9B is an illustration depicting a CD8 complex positioned within a cell membrane in accordance with various embodiments.

FIG. 9C is an illustration depicting a CD4 molecule positioned within a cell membrane in accordance with various embodiments.

FIG. 10A is an illustration depicting a pMHC class I molecule comprising a multi-protein complex associated with a peptide positioned within a cell membrane in accordance with various embodiments.

FIG. 10B is an illustration depicting a pMHC class II molecule comprising a multi-protein complex associated with a peptide positioned within a cell membrane in accordance with various embodiments.

FIG. 11 is a cell pathway process of a peptide-presenting pathway in accordance with various embodiments.

FIG. 12 is an illustration of an exemplary method of generating an MHC complex including a UV cleavable peptide in accordance with various embodiments.

FIG. 13 is an illustration of an exemplary method of exchanging a UV cleavable peptide with a peptide of interest in accordance with various embodiments.

FIG. 14 is a schematic illustration of a system for measuring binding kinetics for assessing live cell activation in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes various embodiments of technologies for assessing ligand (e.g., major histocompatibility complex displaying peptide [pMHC], co-receptors [e.g., CD4, CD8), etc.] and receptor (e.g., T cell receptor [TCR], B cell receptor [BCR], etc.) interactions and kinetics. Such technologies enable researchers and medical practitioners to optimize binding interactions and kinetics to obtain a desired adaptive immune response. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

It should be understood that any uses of subheadings herein are for organizational purposes and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary descriptions of specific features are used, largely for informational purposes, and not in any way to limit the design, subfeature, and functionality of the specifically described feature.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those available and commonly used in the art.

### Definitions

The term “amino acid” as used herein, generally refers to the group of carboxy α-amino acids, which directly or in form of a precursor can be encoded by a nucleic acid. The individual amino acids are encoded by nucleic acids consisting of three nucleotides, so called codons or base-triplets. Each amino acid is encoded by at least one codon. This is known as “degeneration of the genetic code”. The term “amino acid” as used within this application denotes the naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “antigen” can be used interchangeably with “peptide” and can be associated with a major histocompatibility complex (MHC) in accordance with various embodiments. In various embodiments, an MHC can display a peptide for recognition by appropriate lymphocytes (e.g., T cells, B cells). In some embodiments, binding kinetics between an MHC and a receptor (e.g., T cell receptor [TCR] or B cell receptor [BCR]) can change based on properties of a peptide (e.g. peptide sequence, second order structures, third order structures, and higher order structures). In various embodiments, peptides can comprise a polymer of amino acids.

The term “antigen presenting cells” as used herein, generally refers to a cell that can present an antigen in some way. Antigen presenting cells (APCs) can include professional APCs and non-professional APCs.

Professional APCs may specialize in presenting antigens to T cells. In various embodiments, professional APCs may include macrophages, B cells, and dendritic cells. Professional APCs may internalize pathogens or foreign particles (e.g. cancer cells, bacterial cells, etc.) by phagocytosis (e.g. macrophages) or by receptor-mediated endocytosis (B cells). In some situations, pathogens or foreign particles may be processed by proteolysis and resulting peptide fragments (e.g., antigens) may be bound to MHC molecules to form MHC complexes. An MHC complex, including a peptide fragment, can then migrate to the cell membrane and be displayed on its surface for recognition and interaction by a T cell. Professional APCs generally may include co-stimulatory molecules as well as MHC class II. Non-limiting examples of professional APCs include dendritic cells, macrophages, and B cells.

Non-professional APCs may include all nucleated cell types of a subject. In some cases, a non-professional APCs may include an MHC class I molecule coupled to β-2 microglobulin to display endogenous peptides on its cell membrane.

The term “bead,” as used herein, generally refers to a particle. The bead can be a solid or semi-solid particle. The bead can be a gel bead. The gel bead can include a polymer matrix (e.g., matrix formed by polymerization or cross-linking). The polymer matrix can include one or more polymers (e.g., polymers having different functional groups or repeat units). Polymers in the polymer matrix can be randomly arranged, such as in random copolymers, and/or have ordered structures, such as in block copolymers. Cross-linking can be via covalent, ionic, or inductive, interactions, or physical entanglement. The bead can be a macromolecule. The bead can be formed of nucleic acid molecules bound or hybridized together. The bead can be formed via covalent or non-covalent assembly of molecules (e.g., macromolecules), such as monomers or polymers. Such polymers or monomers can be natural or synthetic. Such polymers or monomers can be or include, for example, nucleic acid molecules (e.g., DNA or RNA). The bead can be formed of a polymeric material. The bead can be magnetic or non-magnetic. The bead can be rigid. The bead can be flexible and/or compressible. The bead can be disruptable or dissolvable. The bead can be a solid particle (e.g., a metal-based particle including but not limited to iron oxide, gold or silver) covered with a coating comprising one or more polymers. Such coating can be disruptable or dissolvable.

In various embodiments, a bead may be a sub-category of a solid support. Other solid supports may include surfaces of a plate (e.g., a surface of a well of a 96-well plate), a surface of a vial, a surface of a microscope slide, etc. In some embodiments, surfaces may be chemically treated to interact with receptors and/or co-receptors.

The term “binding affinity,” as used herein, generally refers to the strength of the sum total of non-covalent interactions between a single binding site of a molecule (e.g., TCR or BCR) and its binding partner (e.g., pMHC). Unless indicated otherwise, as used herein, “binding affinity” refers to intrinsic binding affinity which reflects a 1:1 interaction between members of a binding pair (e.g., pMHC and TCR, pMHC and BCR). In various embodiments, affinity of a molecule X for its partner Y can generally be represented by the dissociation constant (Kd). In various embodiments, affinity of a molecule X for its partner Y can generally include a dwell time. In some embodiments, affinity of a molecule X for its partner Y can generally be represented by the dissociation constant (Kd). In some embodiments, affinity of a molecule X for its partner Y can generally include a rate of complex formation. In some embodiments, affinity of a molecule X for its partner Y can generally be represented by the dissociation constant (Kd) and association constant (Ka). In some embodiments, affinity of a molecule X for its partner Y can generally be represented a frequency and duration of interactions.

The term “binding site” is interchangeable with the term “antigen-binding region” and as used herein generally refers to a moiety that can specifically bind to a target (e.g. pMHC). Exemplary binding sites can include peptides, antibody fragments, domain antibodies, or variable domains of single chain antibodies. In some embodiments, TCRs comprise binding sites. In some embodiments, BCRs comprise binding sites. The antigen binding site can be a naturally occurring binding site or an engineered antigen binding site. Exemplary engineered antigen binding sites are DARPINs, domain exchanged antibodies or domain exchanged antibody fragments, and dual variable domain antibodies.

The term “biological particle,” as used herein, generally refers to a discrete biological system derived from a biological sample. The biological particle can be a macromolecule. The biological particle can be a small molecule. The biological particle can be a virus. The biological particle can be a cell or derivative of a cell. The biological particle can be an organelle. The biological particle can be a cell nucleus. The biological particle can be a rare cell from a population of cells. The biological particle can be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle can be a constituent of a cell. The biological particle can be or can include DNA, RNA, organelles, proteins, or any combination thereof. The biological particle can be or can include a matrix (e.g., a gel or polymer matrix) comprising a cell or one or more constituents from a cell (e.g., cell bead), such as DNA, RNA, organelles, proteins, or any combination thereof, from the cell. The biological particle can be obtained from a tissue of a subject. The biological particle can be a hardened cell. Such hardened cell may or may not include a cell wall or cell membrane. The biological particle can include one or more constituents of a cell, but may not include other constituents of the cell. An example of such constituents is a nucleus or an organelle. A cell may be a live cell. The live cell can be capable of being cultured, for example, being cultured when enclosed in a gel or polymer matrix, or cultured when comprising a gel or polymer matrix.

As used herein, the term “biophysical parameter data” can include any measurement or observation of or relating to a biological particle. In some embodiments, biophysical parameter data can comprise any measurement or characteristics of or relating to binding kinetics (e.g. T cell:pMHC and/or B cell:pMHC rates and rate constants).

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

The term “dilution buffer” generally refers to a buffer than can increase a volume of a sample. In various embodiments, dilution buffers can increase a sample volume by 1, 2, 3, 4, 5, 6, 7, 8, 9, 10, 15, 20, 25, 50, 75, or 100 times an original sample volume. In some embodiments, the dilution buffer can include the same chemical properties as a buffer containing the sample.

The terms “fixation agent” and “fixation buffer” are used interchangeably and generally refer to an agent that can keep cells in a particular state for examination. In various embodiments, fixation buffer can prevent deterioration. In some embodiments, fixation buffer can be used in a sampling step. In some embodiments, fixation buffer can be used to stop a reaction (e.g. pMHC and TCR interactions and/or pMHC and BCR interactions). 4% paraformaldehyde (PFA) solution in phosphate-buffered saline (PBS) can be used as a fixation buffer in accordance with some embodiments.

The terms “coupled,” “linked,” “conjugated,” “associated,” “attached,” “connected” or “fused,” as used herein, may be used interchangeably herein and generally refer to one molecule (e.g., polypeptide, receptor, analyte, etc.) being attached or connected (e.g., chemically bound) to another molecule (e.g., polypeptide, receptor, analyte, etc.). In various embodiments described herein, linked comprises covalently linked or non-covalently linked.

As used herein, the term “linker” may be used interchangeably with the term “spacer” and means an inert polymer that can hold two or more molecules together. In various embodiments, linkers may act to distance two molecules away from one another. In various embodiments, a linker length may be selected for a specified application. In various embodiments, linkers may comprise polyethylene glycol (PEG), Poly(N-vinylpyrrolidone) (PVP), Polyglycerol (PG), Poly(N-(2-hydroxypropyl) methacrylamide) (PHPMA), Polyoxazolines (POZs), biotin, avidin, streptavidin or any other known or useful non-reactive molecule able to connect two or one other molecules together. As used herein, linkers may be used to connect oligonucleotides to cleavable substrates.

As used herein, the term “major histocompatibility complex” (“MHC”) generally refers to a group of proteins on a cell surface that play a role in immune response. The function of MHC molecules can be to bind peptide fragments derived from pathogens and display them on the cell's surface. As used herein, the term “pMHC” generally refers to a peptide complexed with MHC. In various embodiments, MHC comprises a MHC Class I. In various embodiments, MHC comprises a MHC Class II. As used herein, the term “ones” means more than one. As used herein, the term “plurality” can be 2, 3, 4, 5, 6, 7, 8, 9, 10, or more. As used herein, the term “detection molecule” generally means anything that can generate a signal or be detected. More specifically, detection molecules can comprise fluorescent molecules. In some embodiments, detection molecules can comprise fluorescent molecules that do not deteriorate when undergoing a fixation process (e.g. one that uses fixation buffer). Detection molecules can be exogenous or endogenous. Detection molecules can be coupled to macromolecular constituents such as proteins, antibodies, aptamers, or amino acids using enzymatic, chemical or other known labeling methods. Detection molecules can be coupled to biotinylated structures (e.g. avidin, streptavidin, biotin). Detection molecules can be coupled to beads. In various embodiments, detection molecules may comprise NHS-ester fluorophores. In many embodiments, NHS-ester fluorophores can be covalently conjugated to solvent exposed surface lysine residues of an amino acid sequence. Such amino acid sequences may be sequences of MHC molecules (e.g., MHC class I or MHC class II). The fluorescent molecules used herein may be selected to measure probes binding to TCRs or BCRs. In various embodiments, fluorophores may be selected based on their resistance to UV radiation. As used herein, the term “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent. The term “variable”, as used in connection with T cell receptors (TCRs) and B cell receptors (BCRs), refers to the fact that certain portions of the TCR and the BCR variable domains differ extensively in sequence among TCRs and BCRs and are used in the binding and specificity of each particular TCR and BCR for its particular peptide (e.g. a peptide associated with MHC class I, a peptide associated with MHC class II). However, the variability is not evenly distributed throughout the variable domains of TCRs and BCRs. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen/pMHC-binding site.

The term “hypervariable region” when used herein refers to the amino acid residues of a TCR, BCR, or antibody which are responsible for antigen/pMHC-binding. The hypervariable region generally comprises amino acid residues from a “complementarity determining region” or “CDR” and/or those residues from a “hypervariable loop.” In some embodiments, “CDR” means a complementary determining region of a TCR, BCR, or antibody. “Framework Region” or “FR” residues are those variable domain residues other than the hypervariable region/CDR residues as herein defined.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo-or polynucleotide chemistry and hybridization described herein are those available and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those available and commonly used in the art.

### I. Overview

Immune systems have high response capacities for detecting abnormalities and protecting the host organism. The high capacity of detection can be achieved through highly variable complementary determining regions (CDRs) of either the α and β chains or δ and γ chains of the TCR interacting with peptide-major histocompatibility complexes (pMHCs). In various embodiments, CDRs combined with the peptide sequence of pMHCs can substantially contribute to TCR:pMHC binding affinity. The assays described herein can quantitatively measure TCR:pMHC and BCR:pMHC binding events in a high-throughput fashion.

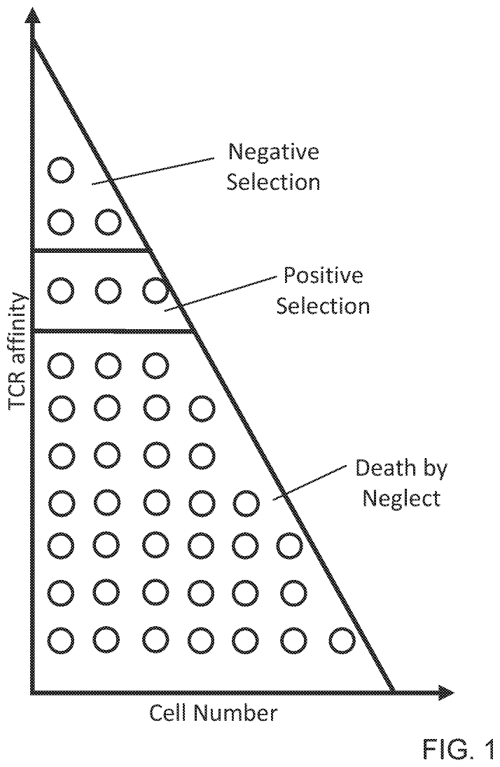

Referring to FIG. 1, the concepts of positive and negative selection of lymphocytes (e.g., T cells) in the Thymus are described. In a T cell maturation process, only a narrow band of T cells are positively selected. When a T cell receptor (TCR) of a T cell has little to no affinity to host/self pMHCs located in the Thymus, those T cells will be negatively selected and suffer a death by neglect (e.g. they will not continue the maturation process). When a T cell has a high affinity for host/self pMHCs located in the Thymus, those T cells will be negatively selected and destroyed via apoptosis. Positively selected T cells include TCRs that can bind to host/self pMHCs within a tolerated affinity range. What is described in FIG. 1 is known as the affinity threshold hypothesis and Thymic selection depends on T cell receptor affinity for self pMHCs. As such, living systems can generate T cells that are largely self-tolerant and still able to identify and remove foreign invaders (e.g., viruses and cancer cells).

In various organisms, TCR diversity can arise from genetic recombination of DNA-encoded segments in individual somatic T cells using a mechanism referred to as somatic V(D)J recombination using RAG1 and RAG2 recombinase. In various embodiments, V(D)J recombination occurs in developing T cells during the early stages of maturation in the Thymus. In some embodiments, V(D)J recombination can be a necessary feature of adaptive immune response. In various organisms BCR diversity can arise using similar recombination methods.

In humans and other mammals V(D)J recombination can occur in the lymphoid organs. In various embodiments, a lymphoid organ can comprise a thymus. In various biological systems, V(D)J recombination can nearly randomly rearrange variable (V), joining (J), and in some cases, diversity (D) gene segments. In various embodiments, V(D)J recombination can result in modification of CDR regions. In some embodiments, V(D)J recombination can result in CDRs comprising novel amino acid sequences.

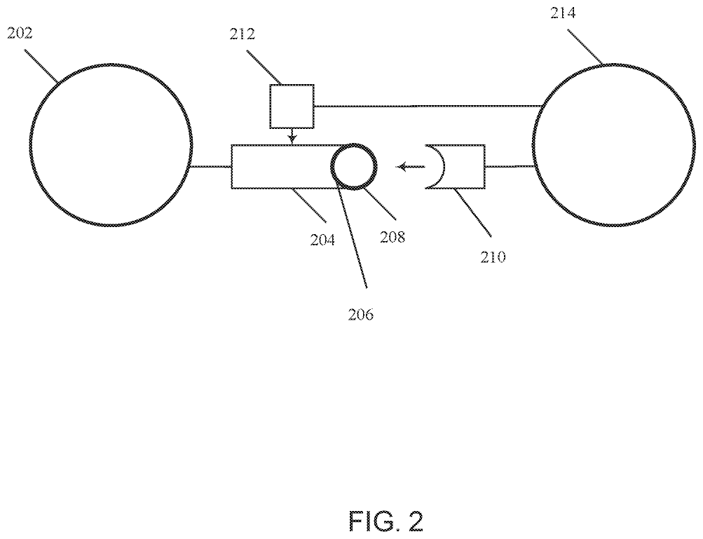

Positively selected T cells can then move throughout a host organism as part of an adaptive immune response. Referring to FIG. 2, interactions between cell surface proteins 210 (e.g., receptors, TCRs) of T cells 214 and cell surface proteins (e.g., an antigenic peptide 208 bound to an antigen-binding region 206 of a polypeptide molecule 204 such as a pMHC) of other cells (e.g., an antigen presenting call 202) help determine whether T cells 214 are positively selected and later be activated to destroy bound cells. In some embodiments, a co-receptor 212 such as CD4 or CD8 of the T cell 214 can bind a region of the polypeptide molecule 204 to aid in the selection process. This interaction may be especially important because it can be involved in T cells' 214 ability to recognize host cells (e.g., self) versus invading or malignant cells (see positive selection above). Specifically, the number of total TCR interactions occurring over a time period can determine the T cell response.

Host cells generally express major histocompatibility complexes (MHCs) on their cell surfaces which each can include a peptide for T cell recognition. Specifically, T cells can include T cell receptor (TCR) on their surfaces that can recognize the MHC molecules of other cells. MHC peptides play a role in determining association rates and dissociation rates (e.g., binding affinity) of the MHC/TCR complex which regulate adaptive immune response.

In natural systems, peptides that bind to MHC molecules can be generated mainly from degradation of cytosolic proteins by the proteasome. Thereafter, the MHC:peptide complex can be inserted into the external plasma membrane via the endoplasmic reticulum. Therefore, MHC molecules function to display intracellular proteins to cytotoxic T cells.

Healthy cells will display peptides from normal cellular protein turnover and T cells will not be activated. However, when a cell expresses foreign proteins (e.g., those originating from a viral infection) TCRs specific for the MHC:peptide complex will recognize and cause cell death.

Cell based assays are useful because some of the parameters governing association rates and dissociation rates can depend on live cell interactions. Non-limiting examples of those dependencies include catch bonds that bind tighter when force is applied and slip bonds that rupture under force. Therefore, it is possible for cell-based assays to perform better than in-vitro assays. Current cell-based options (e.g., the tetramer stain) are often low throughput and do not yield rate constant related data. Specifically, having a single MHC molecule associated with a single fluorophore or known quantity of fluorophores allows quantitative data to include rate binding information because it allows study of pMHC:receptor interactions in the presence of co-receptors within a cell membrane. For these and other reasons, a cell-based assay is ideal for studying the binding kinetics and interactions between pMHC:TCR and/or pMHC:BCR as well as other ligand-receptor binding kinetics and interactions. The present disclosure includes assays and methods for rapidly and accurately measuring data for determining association and dissociation rate constants.

### II. Compositions

The present invention provides for compositions for use in biokinetic assays. An example of a composition may include a polypeptide molecule having an antigen-binding region with one or more fluorophores linked to the polypeptide molecule and an antigenic peptide bound to the antigen-binding region of the polypeptide molecule. Another example of a composition may include a polypeptide molecule comprising an antigen-binding region, one or more fluorophores linked to the polypeptide molecule, and an antigenic peptide bound to the antigen-binding region of the polypeptide molecule, wherein the antigenic peptide comprises a UV cleavable amino acid.

In various embodiments, a polypeptide molecule may include a single polypeptide molecule (e.g., a first polypeptide chain). In alternative embodiments, a polypeptide molecule may include two or more polypeptide molecules. In many embodiments, a polypeptide molecule may include a first polypeptide molecule (e.g., a first polypeptide chain) and a second polypeptide molecule (e.g., a second polypeptide chain).

### Major Histocompatibility Complex (MHC) I

Aspects of a T cell immune response comprise recognition of peptides (e.g., antigens) by T cells. In various embodiments, the role of a major histocompatibility complex (MHC) can be to mobilize peptides to a cell surface for TCR recognition by the T cell (e.g., a cytotoxic T cell).

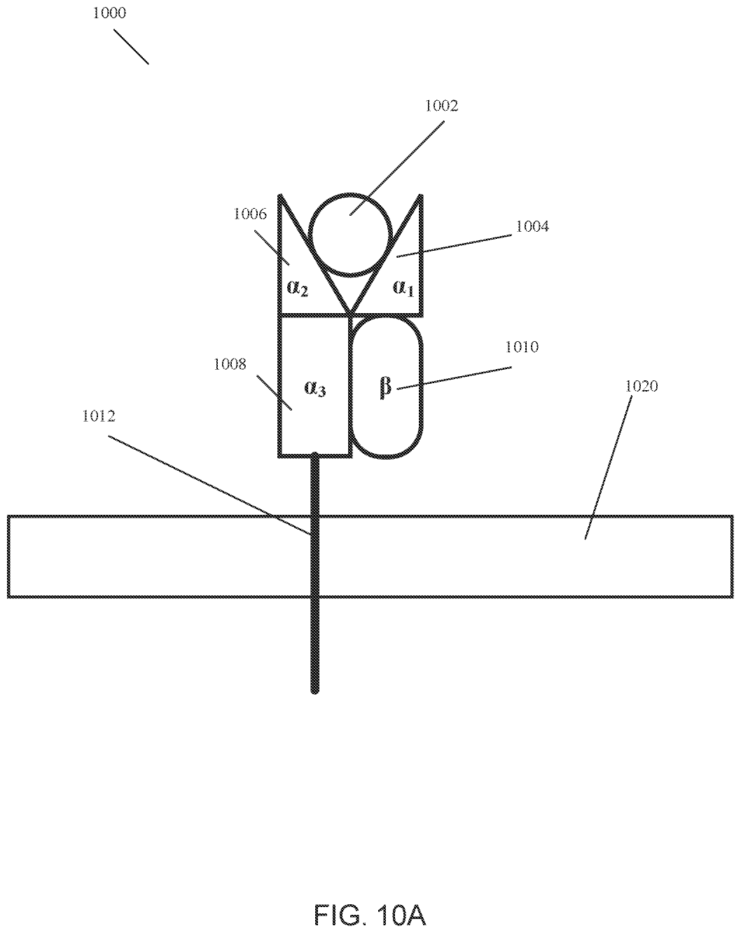

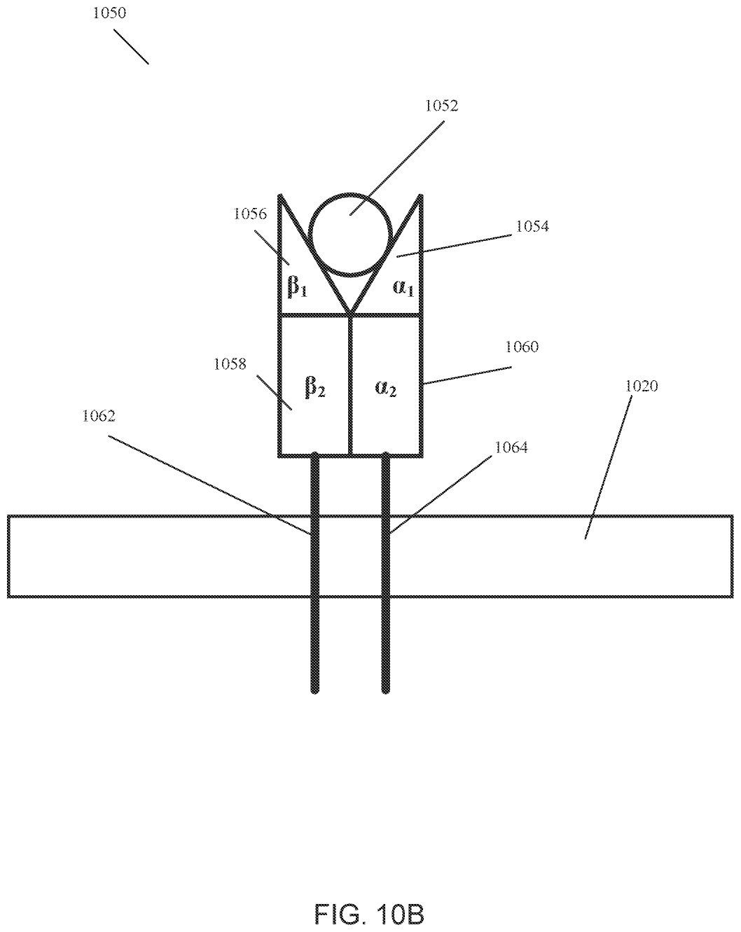

Referring to FIG. 10, a peptide:MHC class I (pMHC I) 1000 is shown embedded within a cell membrane 1020 according to various embodiments. In many embodiments, a pMHC class I 1000 can comprise a first polypeptide chain and a second polypeptide chain. A first polypeptide chain may include an α chain (e.g., α₁ 1004, α₂ 1006, and α₃ 1008). A second polypeptide chain may include a β chain 1010. In various embodiments, pMHC class I 1000 can comprise a peptide 1002 that can serve as a peptide serving as an antigen. In various compositions, the α chain and the β chain 1010 can be associated using non-covalent bonds. In some compositions, the α chain and the β chain 1010 can be associated using covalent bonds. In various compositions, the α chain may comprise an amino acid sequence of a recombinant human leukocyte antigen. In some compositions, the α chain may comprise an amino acid sequence of a recombinant murine histocompatibility system 2.

In various embodiments, an α chain can comprise approximately 350 amino acids and include three globular domains 1004, 1006, 1008. In various embodiments, the three globular domains can be designated α₁ 1004, α₂ 1006, and α₃ 1008.

In various embodiments, the N terminal of an α chain can be located in the α₁ 1004 globular domain. In various embodiments, α₁ 1004 and α₂ 1006 can be located in an extracellular compartment (e.g., within extracellular fluid). In some embodiments, α₁ 1004 and α₂ 1006 can each comprise roughly 90 amino acids. In various embodiments, α₂ 1006 can comprise a loop of 63 amino acids and formation can be facilitated using disulfide bonds. In various embodiments, α₁ and α₂ can interact to form an antigen-binding region of pMHC 1000.

In various compositions, an antigen-binding region may comprise at least a portion of a first polypeptide chain (e.g., an α chain) of a polypeptide molecule. In other compositions, the first polypeptide chain may include an α₁ domain and an α₂ domain.

In various embodiments, an α chain can comprise α₃ 1008 which can include a transmembrane segment 1012 anchoring pMHC 1000 to a cell membrane 1020. In some embodiments, the transmembrane segment 1012 can comprise 26 amino acids. In some embodiments, α₃ 1008 can comprise a disulfide bond enclosing 86 amino acids to form a loop structure. In many compositions, a first polypeptide chain may include an α₃ domain.

In various embodiments, an α₃ 1008 globular domain can interact with a co-receptor (e.g., a CD8 co-receptor of a T cell). In some embodiments, an α₃-co-receptor interaction can hold pMHC class I 1000 in place and a TCR on a cell membrane surface of the T cell can bind α1-α2 heterodimer ligand. In some embodiments, an α3-CD8 interaction can allow the α1-α₂ heterodimer ligand to interrogate the MHC associated peptide for antigenicity. In various embodiments, the C terminal of an α chain can be located in the α₃ 1004 globular domain. In some assays described herein, blocking anti-CD8 antibodies may be used.

The cytoplastic tails of CD8 can interact with Lck (lymphocyte-specific protein tyrosine kinase) and Lck can phosphorylate the cytoplasmic portion of CD3 and ζ-chains (also referred to as a ζ domain herein) of the TCR complex. Phosphorylation of CD3 and the ζ-chains can lead to activation of a variety of transcription factors (e.g., NFAT, NF-κB, and AP-1) that can ultimately affect expression of certain genes downstream of a signaling cascade.

In various compositions, a second polypeptide chain may be linked to a first polypeptide chain as described herein. For example, the second polypeptide chain (e.g., a β chain 1010) may be linked to an α₃ of the first polypeptide. In various embodiments, a β chain 1010 of pMHC class I can comprise a disulfide loop. In various embodiments, β chain 1010 can noncovalently interact with a α₃ 1008 globular domain.

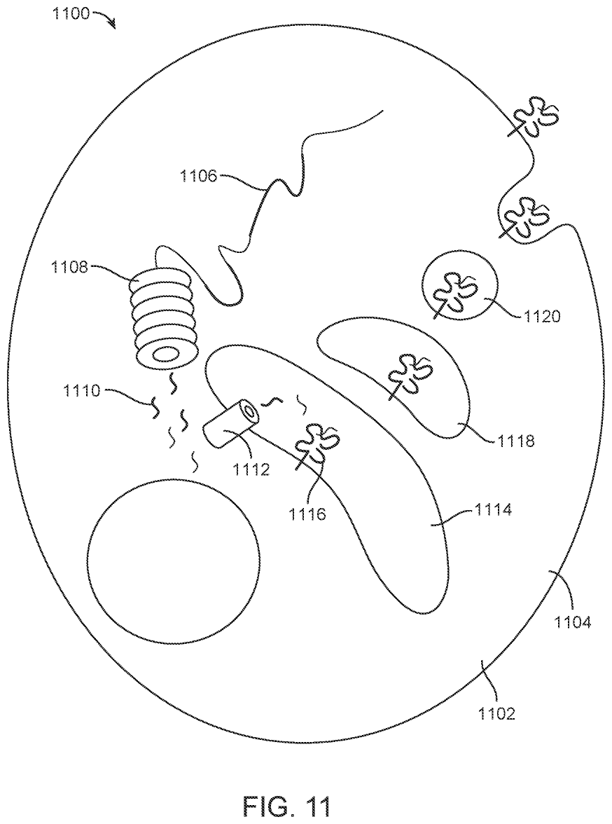

Referring to FIG. 11, an exemplary process of a peptide-presenting pathway 1100. In various embodiments, MHC class I molecules can bind antigen molecules (e.g., peptides) and the entire complex can then be mobilized to a cell surface. In some embodiments, the peptides 1110 can range from eight amino acids to about ten amino acids. In other embodiments, peptides can comprise more than 10 amino acids.

In various embodiments, MHC class I 1116 can bind peptides 1110 derived from intracellular proteins 1106 and display the peptides 1110 for recognition by T cells exterior to the cell 1102. Under some naturally occurring conditions, the source of intracellular proteins 1106 can comprise viral proteins that previously entered the cell. In other naturally occurring conditions, the source of intracellular proteins 1106 can comprise protein derived from infected cells. In alterative situations, the source of intracellular proteins 1106 can comprise protein from cancerous cells. In some embodiments, the source of intracellular proteins 1106 can comprise protein from healthy cells and generally an immune response would not precipitate unless the host organism had an autoimmune disorder or some other ailment.

In various biological systems, a proteasome 1108 comprises protein complexes capable of degrading proteins by proteolysis. In various embodiments, a proteasome 1108 can break peptide bonds of intracellular proteins 1106 using proteases to produce peptides 1110.

In various biological systems, a transporter associated with antigen processing (TAP) 1112 complex can delivery cytosolic peptides into an endoplasmic reticulum 1116. In some embodiments, a TAP 1112 structure can comprise two proteins (e.g., TAP-1 and TAP-2).

In some aspects of a process of a peptide-presenting pathway 1100, TAP 1112 transporters can be associated with a peptide-loading complex. In some embodiments, a peptide-loading complex can comprise β₂ microglobulin (e.g., an MHC chain), calreticulin, ERp57, TAP 1112, tapasin, and an MHC molecule (e.g., MHC Class I). In some embodiments, the peptide-loading complex can hold MHC molecules in place until they have been associated with peptides 1112, thereby, forming pMHC 1116. In various compositions, a second polypeptide chain (e.g., a β chain) may include a sequence of a β₂ microglobulin molecule.

In various embodiments, peptide-presenting pathway 1100 can comprise a secretory pathway where pMHC 1116 can move to a Golgi 1118. In some embodiments, the Golgi 1118 can package pMHC 1116 into secretory vesicles 1120 for further transport.

In various embodiments, secretory vesicles 1120 containing pMHC 1116 can fuse to a cell membrane 1104 of the cell 1102 and present peptides 1110 to the extracellular space for T cell recognition.

### Major Histocompatibility Complex (MHC) II

Aspects of a T cell immune response comprise recognition of peptides (e.g., antigens) by T cells. In various embodiments, the role of a major histocompatibility complex (MHC) can be to mobilize peptides to a cell surface for TCR recognition by the T cell (e.g., a helper T cell).

Referring to FIG. 10B, a peptide-MHC class II (pMHC II) 1050 is shown embedded within a cell membrane 1020 according to various embodiments. In many embodiments, a pMHC class II 1050 can comprise a first polypeptide chain and a second polypeptide chain. A first polypeptide chain may include an α chain (e.g., α₁ 1054 and α₂ 1060). A second polypeptide chain may include a β chain (e.g., β₁ 1056 and β₂ 1058). In various embodiments, pMHC class II 1050 can comprise a peptide 1052 that can serve as a peptide serving as an antigen. In various compositions, the α chain and the β chain can be associated using non-covalent bonds. In some compositions, the α chain and the β chain can be associated using covalent bonds. In various compositions, the α chain may comprise an amino acid sequence of a recombinant human leukocyte antigen.

In various embodiments, an α chain include two globular domains 1054, 1060. In various embodiments, the two globular domains can be designated α₁ 1054 and α₂ 1060.

In various embodiments, a β chain include two globular domains 1056, 1068. In various embodiments, the two globular domains can be designated α₁ 1054 and α₂ 1060.

In various embodiments, the N terminal of an α chain can be located in the α₁ 1054 globular domain. In various embodiments, β₁ 1056 and β₂ 1058 can be located in an extracellular compartment (e.g., within extracellular fluid).

In various compositions, an antigen-binding region may comprise at least a portion of a first polypeptide chain (e.g., an α chain) and at least a portion of a second polypeptide chain (e.g., a β chain) of a polypeptide molecule.

In various embodiments, an α chain can comprise α₂ 1060 which can include a transmembrane segment 1064 anchoring pMHC class II 1050 to a cell membrane 1020. In various embodiments, a β chain can comprise β₂ 1058 which can include a transmembrane segment 1062 anchoring pMHC class II 1050 to a cell membrane 1020.

In various embodiments, a co-receptor (e.g., a CD4 molecule) may interact with a β₂ domain of an MHC class II molecule through its D₁ domain.

The cytoplastic tail of CD4 can interact with Lck (lymphocyte-specific protein tyrosine kinase) and Lck can phosphorylate the cytoplasmic portion of CD3 and ζ-chains of the TCR complex. Phosphorylation of CD3 and the ζ-chains can lead to activation of a variety of transcription factors (e.g., NFAT, NF-κB, and AP-1) that can ultimately affect expression of certain genes downstream of a signaling cascade.

In various compositions, a second polypeptide chain may be linked to a first polypeptide chain as described herein. For example, the second polypeptide chain (e.g., a β chain) may be linked to a first polypeptide (e.g., an α chain).

### T Cell Receptor (TCR)

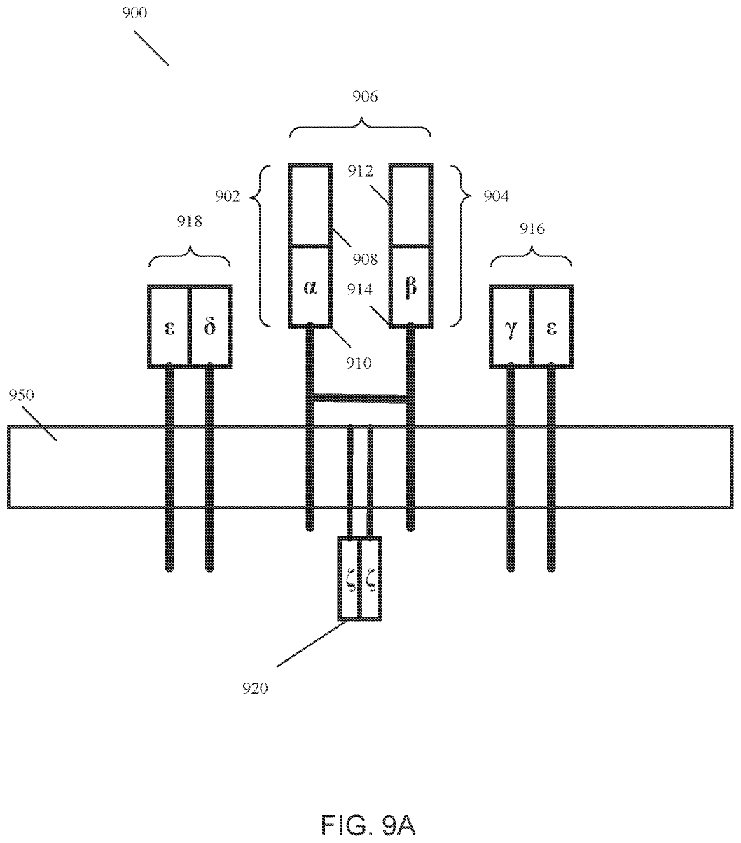

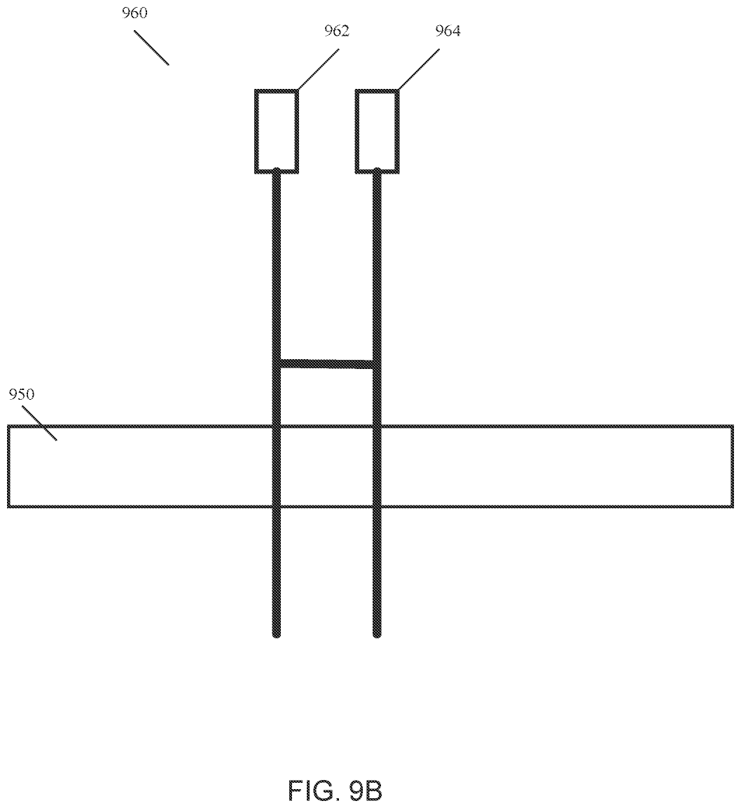

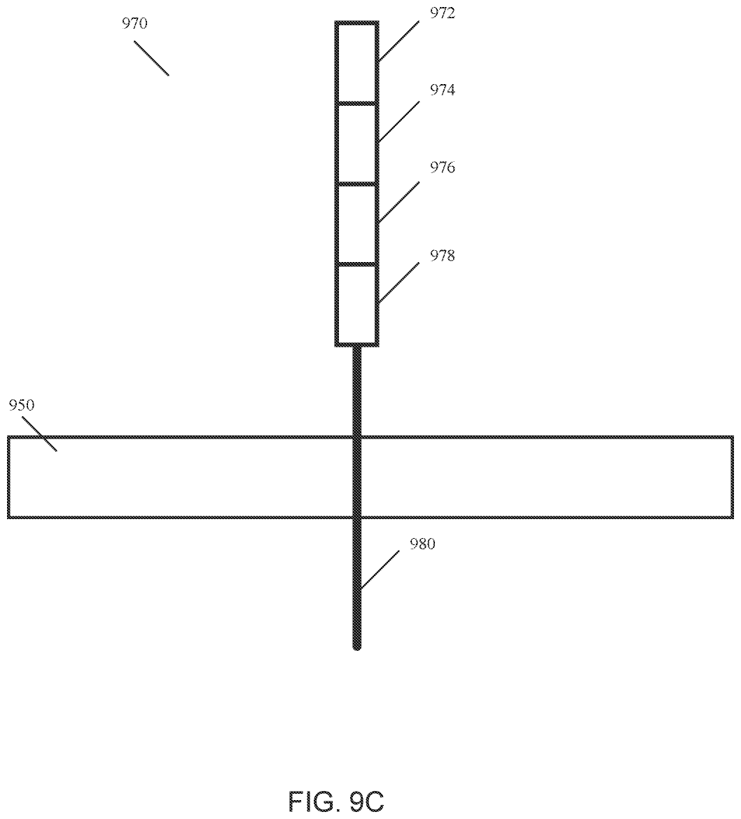

Aspects of an adaptive immune response system comprise T cells including membrane associated TCR. In some embodiments, adaptive immune responses comprise CD28 for providing a co-stimulatory signal. Referring to FIG. 9A, a T cell Receptor (TCR)-CD3 complex 900 is illustrated embedded in a T cell membrane 950. In various embodiments, a TCR-CD3 complex 900 can comprise a disulfide-linked membrane-anchored heterodimeric protein. In many embodiments, the disulfide-linked membrane-anchored heterodimeric protein can comprise an alpha (α) chain 902 and a beta (β) chain 904. In various embodiments, TCR-CD3 complex 900 can comprise an alternate receptor, formed by gamma (γ) and delta (δ) chains. In various embodiments, a TCR-CD3 complex 900 α chain 902 and β chain 904 form the structure of an antigen-binding site (e.g., pMHC binding site 906).

In various T cell conformations, an α chain 902 can comprise two extracellular domains, including a variable region 908 and a constant region 910. In various embodiments, a β chain 904 can comprise two extracellular domains, including a variable region 912 and a constant region 914. In some conformations, the constant regions 910, 914 can be adjacent to a cell membrane 950. In some conformations, the variable regions 908, 912 can form a pMHC binding site 906 and can bind a pMHC.

Each of the TCR chains 902, 904 can comprise a variable region 908, 912 and each variable region 908, 912 can comprise three hypervariable or complementarity-determining regions (CDRs). In some embodiments, CDR1, CDR2, and CDR3 can be arranged non-consecutively on the amino acid sequence of the variable domain 908, 912 of the TCR 900. In some embodiments, CDR3 can be the primary region for recognizing a processed antigenic peptide of a pMHC.

Aspects of immune response can require TCR-CD3 complex 900 to propagate a signal causing T cell activation. In various embodiments, CD3 molecules 916, 918 each have a longer cytoplastic tail than α chain 902 and β chain 904 for allowing signal transduction to occur. In various embodiments, TCR-CD3 complex 900 comprises a first CD3 molecule 916 comprising a γ chain associated with an ε chain. In various embodiments, TCR-CD3 complex 900 comprises a second CD3 molecule 918 comprising a δ chain associated with an ε chain.

In various embodiments, TCR-CD3 complex 900 ζ chains 920 can couple peptide recognition to several intracellular signal-transduction pathways, including, T cell activation.

Referring to FIG. 9B, CD8 960 can form a dimer comprises a pair of CD8 chains 962, 964 embedded in T cell membrane 950. In various embodiments, CD8 960 comprises a CD8-α 962 and a CD8-β 964 chain, thereby, forming a heterodimer. In alternative embodiments, CD comprises two CD8-α chains, thereby, forming a homodimer. In various embodiments, CD8 960 can interact with a pMHC. In various embodiments, a CD8-α 962 chain of CD8 960 can interact with a α₃ globular domain of pMHC. In various embodiments, assays described herein can characterize CD8:pMHC binding characteristics.

In various embodiments, CD8 chains 962, 964 may be linked through a disulfide linkage. Additionally, CD8 chains 962, 964 can each comprise a transmembrane region and a cytosolic region. In various embodiments, CD8:pMHC interactions can be extracellular.

Referring to FIG. 9C, CD4 970 includes four extracellular domains (D₁ 972, D₂ 974, D₃ 976, and D₄ 978). D₁ 972 and D₃ 976 may be variable domains. D₂ 974 and D₄ 978 may be constant domains. In various embodiments, CD4 970 may interact with a β₂ domain of an MHC class II molecule through its D₁ 972 domain. D₁ 972 may include a β-sandwich hold with seven β-strands in 2 β-sheets. A cytoplasmic tail 980 of CD4 970 may interact with tyrosine kinase Lck, thereby, causing a signal cascade.

### III. Cell-Based Ligand-Receptor Kinetic Binding Assay

In various embodiments, the compositions detailed herein can be used in the ligand-receptor binding assays. For example, a monomeric probe may comprise one or more of the polypeptide molecule fluorophore combinations described herein.

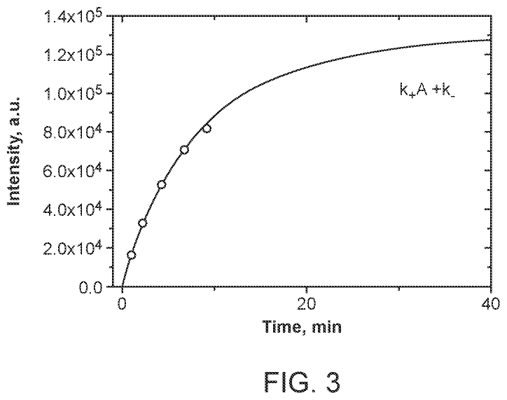

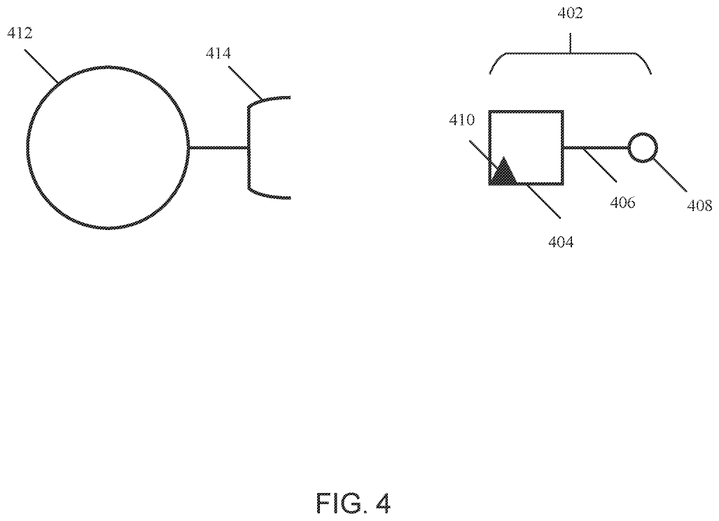

Referring to FIG. 3, a cell-based assay for determining an association or dissociation rate constants is described. What is depicted in FIG. 3 illustrates the general appearance of association time points where signal intensity (e.g., originating from a detection molecule) increases as more monomers bind TCRs. It will be clear to the skilled artisan that the appearance of the curvature will be altered based on whether the graph depicts association or dissociation. FIG. 4 illustrates a schematic of molecules involved in the binding interaction depicted in FIG. 3. Specifically, a T cell with an associated a receptor (e.g., TCR, BRC) can bind to a monomeric probe.

In various embodiments, a cell-based assay for determining an association rate constant may comprise lymphocytes (e.g., T cells, B cells) that are not bound to MHC molecules interacting with monomers (e.g. peptide+MHC+detection molecule) for given time points over a time interval. In some embodiments, the time points may start at 0 minutes and incrementally increase until or after an equilibrium of bound MHC molecules to receptors is reached. In some aspects, taking multiple timepoints between 0 minutes until equilibrium can increase an accuracy of the assay. The curvature data can be processed to determine an association rate constant representative of living cells.

In various embodiments, a cell-based assay for determining a dissociation rate is described. lymphocytes can interact with monomers (e.g., peptide+MHC+detection molecule) until an equilibrium state has been reached (e.g., the rate of associate is equal to the rate of dissociation). In some embodiments, an agent can be applied that can remove monomers from lymphocytes over time interval. Non-limiting examples of removal agents can comprise dilution buffer, chemical agents, environmental conditions (e.g., temperature).

In various embodiments, samples may be collected at time points over the time interval. In some embodiments, timepoints may include between equilibrium (e.g., 0 minutes) and no detectable monomers. In some embodiments, timepoints may include between equilibrium (e.g., 0 minutes) and a second equilibrium. In some embodiments, the second equilibrium can comprise a decreased rate of receptor/MHC interactions. In some aspects, taking multiple timepoints between 0 minutes (e.g., first equilibrium) until second equilibrium can increase an accuracy of the assay. The curvature data can be processed to determine an association rate constants representative of living cells.

### Monomeric Probes

Currently, tetramer probes can be used in a tetramer stain assay. Tetramers offer increased binding avidity to T cells by virtue of being able to bind multiple T cell receptors. The property of increased binding avidity results in increased detection capabilities. The problem is that allowing a probe to bind multiple TCRs at the same time means information involving pMHC:TCR live cell binding kinetics (e.g., information about bimolecular 1TCR 1pMHC) will be lost.

Aspects of the disclosure describe monomeric probes suitable for cell-based kinetic assays in accordance with various embodiments. In various embodiments, monomeric probes can enable interrogation of rate binding properties (e.g., association and dissociation rate constants) by virtue of each probe being able to bind only one receptor (e.g., TCR, BCR). Including a 1:1 (pMHC:receptor) binding ratio can allow fluorescence to correspond with a number of pMHC:receptor binding events. Importantly, the assay described herein making use of monomeric probes can obtain kon and koff for a bimolecular reaction (1receptor and 1ligand (e.g., a pMHC molecule). As such, various assays described herein can comprise monomeric probes.

Aspects of the disclosure include embodiments for monomeric probes used in the various cell-based assays described herein. In addition to the composition described herein, an additional non-limiting example of a monomeric probe 402 is depicted in FIG. 4. The purpose of monomeric probes 402 can be to mimic an antigen presenting complex (e.g., pMHC) on cells and bind to T cells 412 capable of recognizing the antigen (e.g., peptide).

In various embodiments, monomeric probes can comprise a detection molecule 408 linked to an MHC molecule 404.

In various embodiments, MHC molecules 404 can comprise class I MHC molecules. In various embodiments, MHC molecules 404 can comprise class II MHC molecules. In various embodiments, an MHC molecule 404 can bind a peptide 410. In various embodiments, the properties of a peptide can contribute to determining binding kinetics between the MHC molecule 404 and the receptor molecule 414. In some embodiments, the binding kinetics of MHC molecule 404 and receptor molecule 414 can determine whether a T cell 412 is activated and causes cell death for the cell that could be associated with MHC molecule 404.

In various embodiments, an MHC molecule 404 can be linked to a detection molecule 408 through a linker 406. In various embodiments, a linker 406 may include a lysine residue and a detection molecule 408 may comprise a fluorophore bound to the lysine residue as described herein, including in the Examples section.

In some embodiments, coupling can be direct or through an intermediary. In various embodiments, a detection molecule 408 can comprise avidin or streptavidin and an MHC molecule 404 can comprise biotin. In various embodiments, a detection molecule 408 can comprise biotin and an MHC molecule 404 can comprise avidin or streptavidin. In some aspects, binding of biotin to streptavidin causing formation of a monomeric probe 402. In the described portion above, biotin and (streptavidin or avidin) can comprise a linker 406. In some embodiments, a linker 406 can comprise a single molecule. In other embodiments, a linker 406 can comprise more than one molecule (e.g., biotin and (streptavidin or avidin)).

Non-limiting examples of detection molecules 408 can comprise fluorophores, quenching agents Förster resonance energy transfer systems (e.g., FRET), or other molecules that can be quantifiably interrogated.

In various embodiments, a composition for use in a biokinetic assay may comprise a polypeptide molecule comprising an antigen-binding region, one or more fluorophores linked to the polypeptide molecule, and an antigenic peptide bound to the antigen-binding region of the polypeptide molecule.

In various embodiments, a composition for use in a biokinetic assay may comprise a polypeptide molecule comprising an antigen-binding region, one or more fluorophores linked to the polypeptide molecule, and an antigenic peptide bound to the antigen-binding region of the polypeptide molecule, wherein the antigenic peptide comprises a UV cleavable amino acid.

In some embodiments, a polypeptide molecule comprises a single polypeptide chain. In other embodiments, a polypeptide molecule may comprise a first polypeptide chain and a second polypeptide chain.

In various embodiments, an antigen-binding region of a polypeptide molecule may comprise at least a portion of the first polypeptide chain of the polypeptide molecule.

In some embodiments, a first polypeptide chain may comprise an α₁ domain and an α₂ domain. In some embodiments, the first polypeptide chain may comprise an α₃ domain.

In various embodiments, a first polypeptide chain may comprise an amino acid sequence of a recombinant human leukocyte antigen.

In various embodiments, a first polypeptide chain may comprise an amino acid sequence of a recombinant murine histocompatibility system 2.

In various embodiments, a second polypeptide chain may be linked to an α₃ domain of a first polypeptide chain.

In various embodiments, a second polypeptide chain comprises an amino acid sequence of a β₂-microglobulin molecule.

In various embodiments, an antigen-binding region comprises at least a portion of a first polypeptide chain and at least a portion of a second polypeptide chain.

In various embodiments, a portion of the first polypeptide chain comprises an α₁ domain, the portion of the second polypeptide chain comprises a β₁ domain, and the α₁ domain and the β₁ domain form the antigen-binding region. In some embodiments, the first polypeptide chain further comprises an α₂ domain. In some embodiments, the second polypeptide chain further comprises a β₂ domain. In many embodiments, the first polypeptide chain comprises an amino acid sequence of a recombinant human leukocyte antigen. In many embodiments, the second polypeptide chain comprises a polypeptide sequence of a recombinant human leukocyte antigen.

In various embodiments, an antigen-binding region comprises at least one α-helix. In some embodiments, the antigen-binding region comprises at least one β-sheet.

In various embodiments, one or more fluorophores are covalently linked to a polypeptide molecule. In some embodiments, the covalent linkage comprises an ester. In various embodiments, the one or more fluorophores are covalently linked to one or more solvent exposed surface lysine residues of the polypeptide molecule.

In various embodiments, first and second polypeptide chains are non-covalently linked. In alternative embodiments, first and second polypeptide chains are covalently linked.

In various embodiments, a polypeptide molecule may be bound to an antigen presenting cell surrogate. In various embodiments, the antigen presenting cell surrogate comprises a bead. In various embodiments, the polypeptide molecule is attached to the antigen presenting cell surrogate by a linker. In some embodiments, the linker comprises a polyethylene glycol (PEG) molecule.

In many embodiments, the composition further comprising a receptor bound to the antigen-binding region of the polypeptide molecule.

In various embodiments, a composition comprises a live lymphocyte. In some embodiments, at least a portion of a receptor is on a cell membrane of the live lymphocyte. In some embodiments, a co-receptor may be located on the cell membrane of the live lymphocyte. In some embodiments, the co-receptor is bound to a portion of the polypeptide molecule. In various embodiments, the live lymphocyte is a T cell and the receptor is a T cell receptor (TCR). In various embodiments, the co-receptor comprises a CD8 molecule. In other embodiments, the live lymphocyte is a B cell and the receptor is a B cell receptor (BCR). In some embodiments, the co-receptor comprises a CD4 molecule. In additional other embodiments, the live cell comprises a macrophage and the receptor comprises a chemokine receptor. In additional other embodiments, the live cell comprises a dendritic cell and the receptor comprises a pattern recognition receptor (PRR).

In various embodiments of the assay, naturally occurring peptides 410 can be interrogated. In some embodiments, artificially created (e.g., synthesis, expression vector, etc.) peptides 410 can be interrogated. Skilled artisans will appreciate that the embodiments herein can benefit from existing peptide libraries. Additional embodiments can include manufacture of peptide libraries.

Aspects of the described systems and methods can involve assaying a variety of different receptors. In various embodiments, peptides may be selected based on a specific receptor being assayed. For example, several peptides are known to bind to OT-1 TCR at different strengths. As such, in some embodiments, known peptide sequences can be assayed.

In various embodiments, manufactured peptides can be assayed. A non-limiting example of a process for manufacturing peptides can comprise solid-phase peptide synthesis (SPPS). In some embodiments, SPPS can assemble peptide chains through successive reactions of amino acid derivatives on a support (e.g., an insoluble porous support).

In some embodiments, peptides 410 with characterized binding properties can be deployed for therapies based on the characterization. In other embodiments, research and development can benefit from being able to deploy peptides 410 having characterized binding properties.

In various embodiments, an antigenic peptide has a length that ranges from 8-11 amino acid residues. In other embodiments, the antigenic peptide has a length that ranges from 15-24 residues. In some embodiments, the antigenic peptide comprises a UV moiety.

### Reaction Mixtures

The present invention provides reaction mixtures for generating probe complexes for use in a biokinetic assays. In various embodiments, a reaction mixture may comprise one or more of the compositions described herein and a target antigenic peptide. In various reaction, the target antigenic peptide may be present at a concentration in a molar excess compared to the antigenic peptide bound to the antigen-binding region of the polypeptide molecule. In various reaction mixtures, the concentration of the target antigenic peptide is 25 times greater than the concentration of the antigenic peptide. Various reaction mixtures may comprise 25 mM TRIS. Various reaction mixtures may comprise a pH of 8.0. Various reaction mixtures may comprise 150 mM NaCl. Various reaction mixtures may comprise 4 mM EDTA. Various reaction mixtures may comprise 5% ethylene glycol.

### Instrumentation Systems

Aspects of the disclosure can benefit from instrumentation systems for collecting quantitative data. In various embodiments, analytes (e.g., ligand-receptor) can be labeled with a detection molecule that can be detectable by an instrumentation system. In various embodiments, instrumentation systems can comprise detection systems. In various embodiments, instrumentation systems can comprise optical system components. In various embodiments, instrumentation systems can comprise fluidic system components. In various embodiments, instrumentation systems can comprise control system components.

The methods described herein can be practiced on any instrumentation system capable of quantifiably analyzing binding kinetics between a ligand and a receptor (e.g., measuring a signal intensity from a detection molecule). Some embodiments benefit from high-throughput systems comprising flow cytometry.

Flow cytometers are designed to analyze large populations of cells by passing the cells through a detection region at very high rates. In the accompanying methods, populations of cells can be prepared for interrogation at varying timepoints along a time interval to generate an association or dissociation curve. A large quantity of binding kinetic data can be necessary for accurately characterizing rate constants contributing to the curve. As such, monomeric probes (e.g., a pMHC and an associated detection molecule) bound to TCRs of T cells can be well suited for flow cytometry analysis.

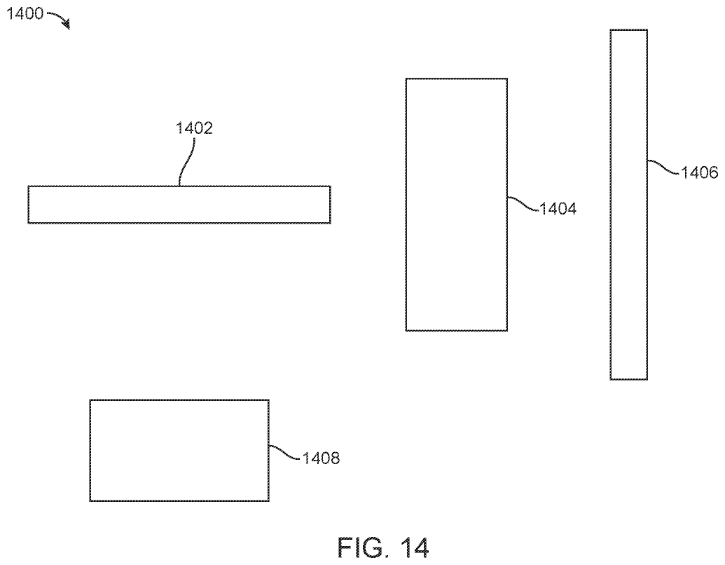

FIG. 14 is a schematic illustration of a system for measuring binding kinetics for assessing live cell activation in accordance with various embodiments. In various embodiments, the system comprises an analytical device 1400 comprising an analysis chamber 1404, wherein the analysis chamber 1404 comprises a composition described herein. The present invention provides systems for measuring binding kinetics for assessing live cell activation, comprising an analytical device 1400 comprising an analysis chamber 1404, wherein the analysis chamber 1404 comprises a reaction mixture described herein. In various systems, the analysis device 1400 comprises an optical source 1402 for interrogating the analysis chamber 1404 and a detector 1406 for detecting a signal.

In various embodiments, a computer system 1408 comprising a non-transitory computer readable storage medium, wherein the non-transitory computer readable storage medium comprises instructions for analyzing the signal.

In various embodiments, analyzing a signal comprises normalizing a signal to a previously determining calibration value.

In various embodiments, an analysis chamber 1404 comprises a flow cell.

In various embodiments, an analytical device 1400 comprises a flow cytometer. In various embodiments, an analytical device 1400 comprises a fluorometer. In various embodiments, an analytical device 1400 comprises a microscope. In various embodiments, an analytical device 1400 comprises a mass spectrometer.

### Methods—Generating a Monomeric Probe Complex

The present invention provides methods of generating monomeric probe complexes. Various methods comprise contacting a polypeptide molecule comprising an antigen binding region with a first antigenic peptide to generate an antigen-presenting complex, contacting the antigen-presenting complex with a plurality of fluorophore molecules to generate a fluorophore-labeled antigen-presenting complex, determining a concentration of fluorophore molecules covalently bound to the fluorophore-labeled antigen-presenting complex, and exchanging the first antigenic peptide with a second antigenic peptide to generate the monomeric probe complex.

In various embodiments, a method of generating a monomeric probe complex comprises a step of exchanging a first antigenic peptide with a second antigenic peptide comprises cleaving the first antigenic peptide to generate a cleaved first antigenic peptide.

In various embodiments, a method of generating a monomeric probe complex comprises a step of cleaving a first antigenic peptide comprises applying UV radiation.

In various methods, a second antigenic peptide has a higher affinity for an antigen binding region than a cleaved first antigenic peptide.

In various methods, UV radiation comprises a wavelength of 365 nanometers.

In various embodiments, a method of generating a monomeric probe complex comprises a step of contacting an antigen-presenting complex with a plurality of fluorophore molecules to generate a fluorophore-labeled antigen-presenting complex comprises covalently linking one or more solvent exposed surface lysine residues of the polypeptide molecule to one or more of the fluorophore molecules.

In various embodiments, a method of generating a monomeric probe complex comprises a step of separating one or more unconjugated fluorophores from the labeled monomeric probe complex.

In various embodiments, a method of generating a monomeric probe complex comprises determining a concentration of a plurality of labeled monomeric probe complexes.

In various embodiments, a method of generating a monomeric probe complex comprises determining an average number of fluorophores conjugated to each of a plurality of the labeled monomeric probe complexes. In some embodiments, the step of determining the average number of fluorophores conjugated to each of the plurality of the labeled monomeric probe complexes includes using a plurality of relative abundance values, wherein each relative abundance value corresponds to a different number of conjugated fluorophores. In some embodiments, each of the plurality of relative abundance values are determined using mass spectrometry.

### Methods—Association

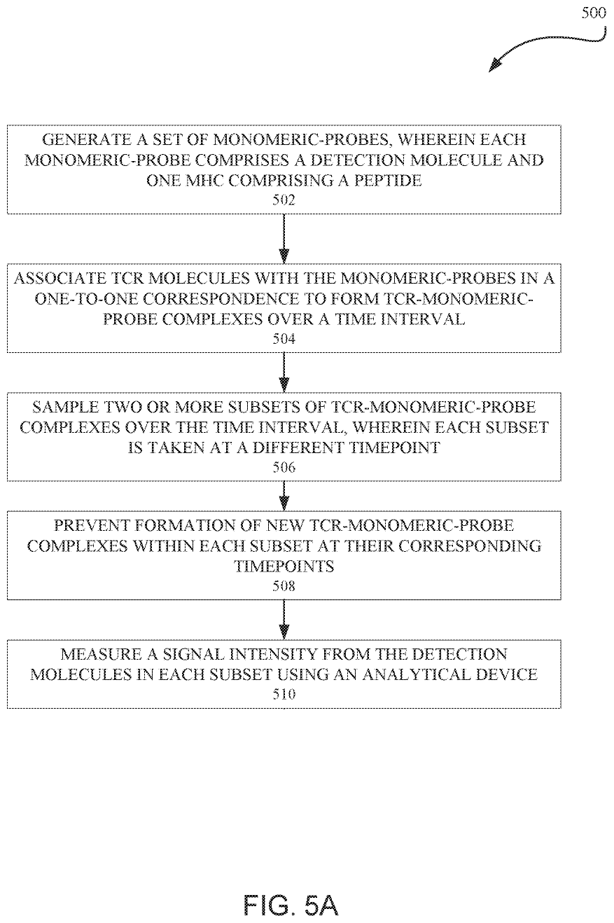

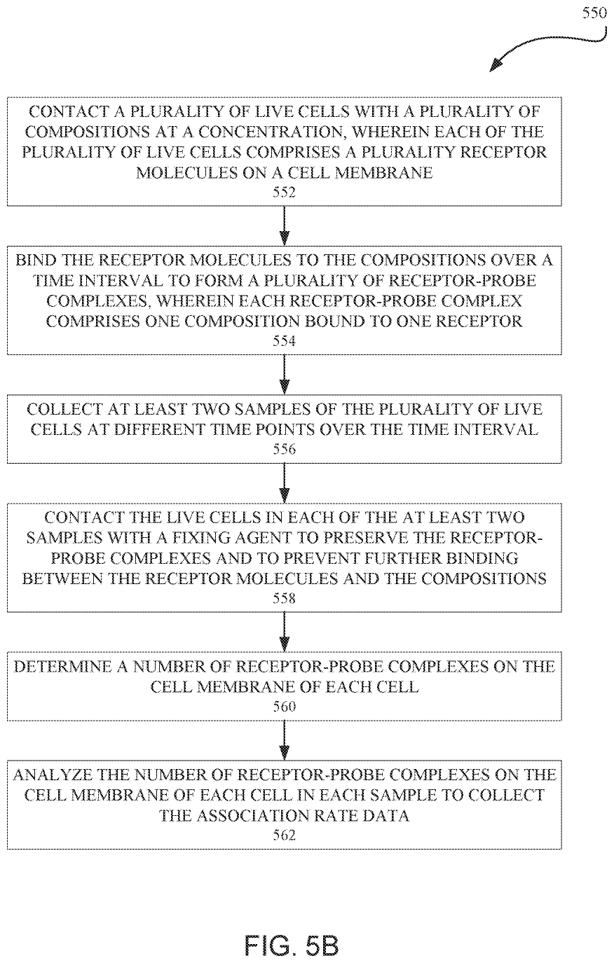

FIG. 5A is a flow chart of a process 550 for collecting association rate data for assessing live cell activation in accordance with various embodiments.

Step 552 comprises contacting a plurality of live cells with a plurality of compositions at a concentration, wherein each of the plurality of live cells comprises a plurality receptor molecules on a cell membrane. In various embodiments, the live cells comprise a T cell. In various embodiments, the live cells comprise a B cell. In various embodiments, the live cells comprise a macrophage. In various embodiments, the live cells comprise a dendritic cell.

Step 554 comprises binding the receptor molecules to the compositions over a time interval to form a plurality of receptor-probe complexes, wherein each receptor-probe complex comprises one composition bound to one receptor. In some embodiments, each of the receptor-probe complexes comprises a CD8 molecule. In some embodiments, each of the receptor-probe complexes comprises a CD4 molecule.

Step 556 comprises collecting at least two samples of the plurality of live cells at different time points over the time interval.

Step 558 comprises contacting the live cells in each of the at least two samples with a fixing agent to preserve the receptor-probe complexes and to prevent further binding between the receptor molecules and the compositions. In various embodiments, the fixing agent comprises paraformaldehyde.

Step 560 comprises determining a number of receptor-probe complexes on the cell membrane of each cell.

Step 562 comprises analyzing the number of receptor-probe complexes on the cell membrane of each cell in each sample to collect the association rate data.

In various embodiments, a signal intensity of the composition is measured using an analytical device. In various embodiments, the analytical device comprises a flow cytometer. In some embodiments, the analytical device comprises a fluorometer.

In various embodiments, the process may include preventing receptor internalization by decreasing a temperature of the receptor-probe complexes. In many embodiments, the temperature is decreased to 4° C.

In various embodiments, the process may include separating one or more unbound compositions from the plurality of live cells.

FIG. 5B is a flowchart of a process 500 for collecting biophysical parameter data for assessing a T cell receptor (TCR) and a peptide-major histocompatibility complex (pMHC) association rate in accordance with some embodiments.

In various embodiments, the TCR molecule can be expressed on a T cell. For non-limiting examples, TCR molecules can be located in, at, or near a cell membrane of the T cell.

Step 502 comprises generating a set of monomeric probes, wherein each monomeric probe comprises a detection molecule and one MHC comprising a peptide.

Step 504 comprises associating TCR molecules with the monomeric probes in a one-to-one correspondence to form TCR-monomeric-probe complexes over a time interval. In some embodiments, the time interval can begin prior to an equilibrium state of TCR-monomeric-probe complexes.

Step 506 comprises sampling two or more subsets of TCR-monomeric-probe complexes over the time interval, wherein each subset is taken at a different timepoint.

Step 508 comprises preventing formation of new TCR-monomeric-probe complexes within each subset at their corresponding timepoints. In some aspects, the step of preventing can include use of a fixation buffer. Fixation buffers can prevent reactions from moving forward in accordance with some embodiments. In some embodiments, a 4% PFA solution in PBS can be used as a fixation buffer. In some situations, embodiments can comprise decreasing a temperature following the preventing step. This added step to the protocol can further prevent formation and may serve to preserve samples. In some embodiments, the temperature can be 4 degrees C.

Step 510 comprises measuring a signal intensity from the detection molecules in each subset using an analytical device.

In various embodiments, a detection molecule comprises a fluorescent molecule. In some embodiments, a detection molecule can comprise a quenching agent. In various embodiments, detection molecule and MHC can be coupled with a linker. In some embodiments, the linker comprises a biotinylated structure. In some embodiments, linker can comprise one, two, three, four, five, six, or any number of molecules. A non-limiting example of a multi-molecule linker comprises biotin bound to avidin or streptavidin. In alternative embodiments, the linker comprises PEG.

In various embodiments, an analytical device comprises a flow cytometer. In some embodiments, the analytical properties of flow cytometers can be suitably applied. A non-limiting example of an analytical property can comprise high throughput capabilities by running liquid carrying cells though a flow cell at a high rate of speed.

In various embodiments, the method can further comprise the step of separating unbound monomeric probes from the TCR molecules. A non-limiting example can comprise one or more wash steps.

### Methods—Dissociation

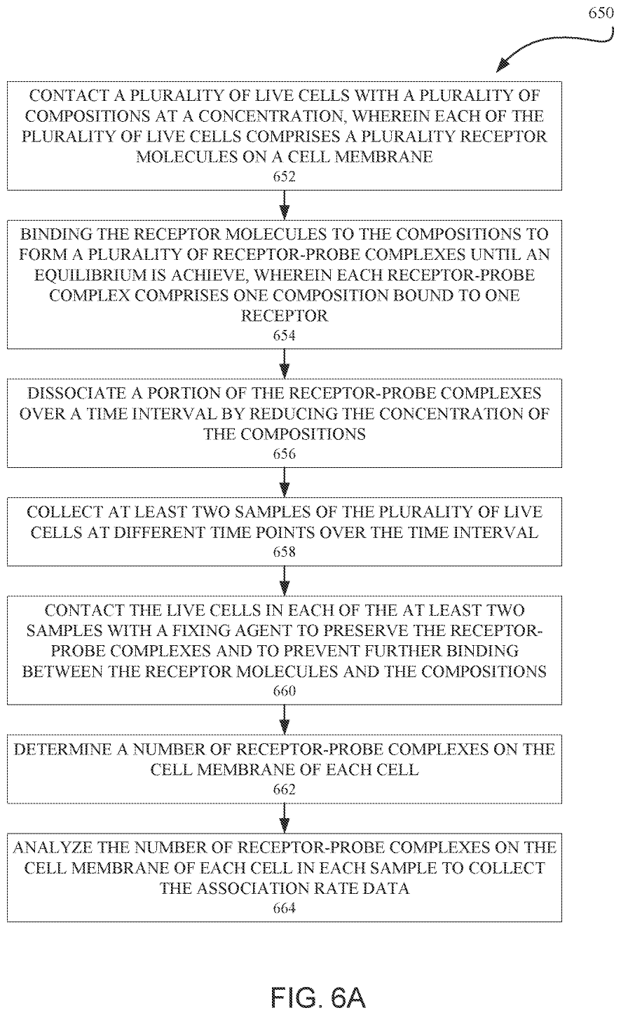

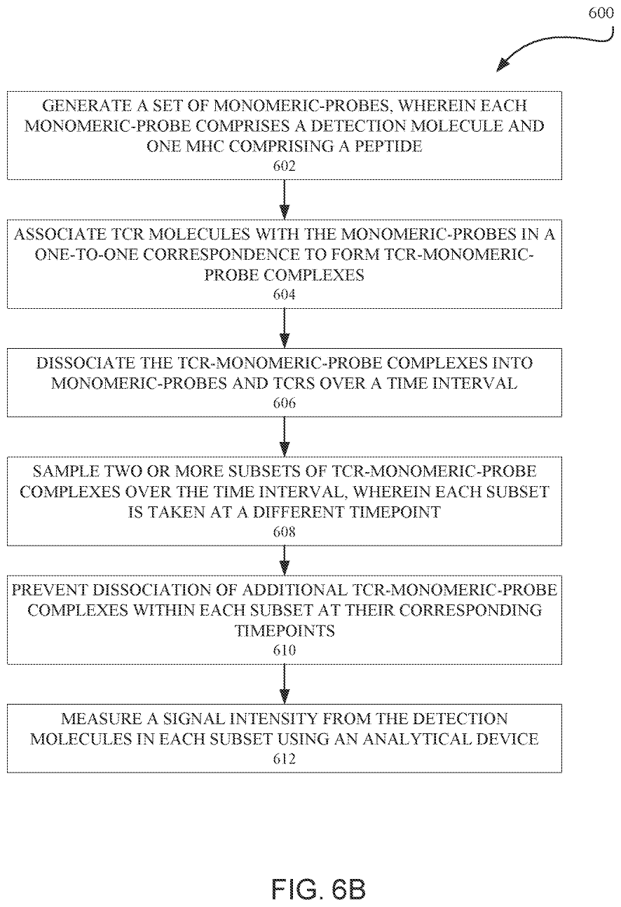

FIG. 6A is a flow chart of a process 650 for collecting dissociation rate data for assessing live cell activation in accordance with various embodiments.

Step 652 comprises contacting a plurality of live cells with a plurality of compositions at a concentration, wherein each of the plurality of live cells comprises a plurality receptor molecules on a cell membrane. In various embodiments, the live cells comprise a T cell. In various embodiments, the live cells comprise a B cell. In various embodiments, the live cells comprise a macrophage. In various embodiments, the live cells comprise a dendritic cell.

Step 654 comprises binding the receptor molecules to the compositions to form a plurality of receptor-probe complexes until an equilibrium is achieve, wherein each receptor-probe complex comprises one composition bound to one receptor. In some embodiments, each of the receptor-probe complexes comprises a CD8 molecule. In some embodiments, each of the receptor-probe complexes comprises a CD4 molecule.

Step 656 comprises dissociating a portion of the receptor-probe complexes over a time interval by reducing the concentration of the compositions.

Step 658 comprises collecting at least two samples of the plurality of live cells at different time points over the time interval.

Step 660 comprises contacting the live cells in each of the at least two samples with a fixing agent to preserve the receptor-probe complexes and to prevent further binding between the receptor molecules and the compositions. In various embodiments, the fixing agent comprises paraformaldehyde.

Step 662 comprises determining a number of receptor-probe complexes on the cell membrane of each cell.

Step 664 comprises analyzing the number of receptor-probe complexes on the cell membrane of each cell in each sample to collect the association rate data.

In various embodiments, a signal intensity of the composition is measured using an analytical device. In various embodiments, the analytical device comprises a flow cytometer. In some embodiments, the analytical device comprises a fluorometer.

In various embodiments, the process may include preventing receptor internalization by decreasing a temperature of the receptor-probe complexes. In many embodiments, the temperature is decreased to 4° C.

In various embodiments, the process may include separating one or more unbound compositions from the plurality of live cells.

Referring to FIG. 6B, a method for collecting biophysical parameter data for assessing a T cell receptor (TCR) and a peptide-major histocompatibility complex (pMHC) dissociation rate is depicted in accordance with some embodiments. In various embodiments, a TCR molecule can be expressed on a T cell.

Step 602 comprises generating a set of monomeric probes. See “Monomeric Probes” section for exemplary embodiments of various monomeric probes. In some embodiments, each monomeric probe comprises a detection molecule and one MHC comprising a peptide. In various embodiments, the detection molecule can comprise a fluorescent molecule. In some embodiments, the detection molecule and MHC can be coupled with a linker. Non-limiting examples of linkers can comprise biotinylated structures including biotin, avidin, and streptavidin. In alternative examples, a linker can comprise PEG.

Step 604 comprises associating TCR molecules with the monomeric probes in a one-to-one correspondence to form TCR-monomeric-probe complexes. In some embodiments, TCR molecules can comprise T cell receptor molecules.

Step 606 comprises dissociating the TCR-monomeric-probe complexes into monomeric probes and TCRs over a time interval. In various embodiments, the time interval can begin at an equilibrium state of TCR-monomeric-probe complexes. In some embodiments, TCR molecules and monomeric probes can be pre-incubated to achieve and equilibrium state. The method can further comprise use of a buffer agent to dilute a solution comprising the TCR-monomeric-probe complexes, thereby, causing dissociation. In come embodiments, the buffer agent can comprise a dilution buffer.

Step 608 comprises sampling two or more subsets of TCR-monomeric-probe complexes over the time interval, wherein each subset is taken at a different timepoint.

Step 610 comprises preventing dissociation of additional TCR-monomeric-probe complexes within each subset at their corresponding timepoints. In some aspects, the step of preventing can comprise use of a fixation buffer. In some embodiments, the method can further comprise the step of decreasing a temperature following the preventing step. A non-limiting example of a temperature an comprise 4 degrees C.

Step 612 comprises measuring a signal intensity from the detection molecules in each subset using a high-throughput analytical device. In various embodiments, the analytical device can comprise a flow cytometer.

Embodiments of the method can comprise the additional step of separating unbound monomeric probes from the TCR molecules. In some embodiments, separating can comprise washing

### EXAMPLES

### Example 1: Components of Probe Complex

Recombinant human β₂-microglobulin (B2M). B2M binds with human leukocyte antigen (HLA) or murine Histocompatibility system 2 to form a non-covalent complex through interactions with the α₃ domain. The resulting heterodimer was major histocompatibility complex (MHC) class I. There was enough sequence homology to use human B2M for both human and murine MHCs.

Recombinant human leukocyte antigen (HLA) or recombinant murine Histocompatibility system 2 (H-2) (α chain of MHC class I complex). When complexed with β₂-microglobulin, the α₁ and α₂ domains form a peptide binding groove, capable of non-covalently binding specific peptide 8-11 amino acids in length.

Peptides. Synthetic 8-11mer peptides were derived from human, murine, chicken, or viral proteins. The peptides bind to peptide binding groove of α₁ and α₂ domains of HLA in complex with β₂-microglobulin with nanomolar to micromolar affinity. Peptide binding to MHC class I stabilizes the recombinant non-covalent complex. Epitope peptide binds to MHC class I to form the antigen recognized by T cell receptors (TCRs).

N-hydroxysuccinimde (NHS)-ester fluorophore. When added in a molar excess in optimal reaction conditions, the NHS-ester fluorophore (Alexa Fluor™ 488 NHS Ester) covalently conjugates to solvent exposed surface lysine residues of MHC class I. Enables fluorescent measurements of probe binding to TCRs. NHS-ester fluorophores were selected based on UV resistance.

### Example 2: Starting Components for Method of Probe Synthesis

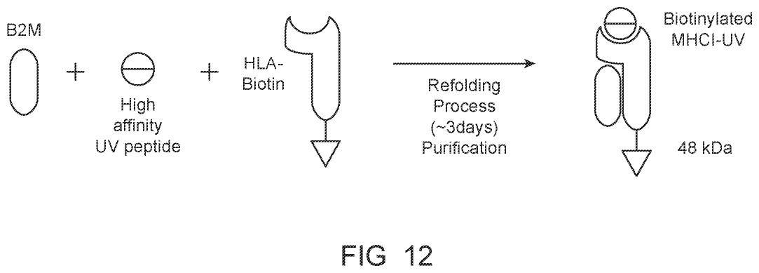

UV-MHC class I or peptide-MHC class I. Recombinant MHC class I was refolded with either a high affinity 8-11 oligomer peptide including a non-natural, UV cleavable, amino acid or with the epitope peptide of interest. Peptides were either obtained commercially or produced in-house. Peptides, for example, in Grotenbreg, Gijsbert M., et al., “Discovery of CD8+ T cell epitopes in Chlamydia trachomatis infection through use of caged class I MHC tetramers.” PNAS, 2008. Vol. 105, no. 10, pages 3831-3838, the disclosure of which is incorporated herein by reference in its entirety. The process is shown pictorially in FIG. 12.

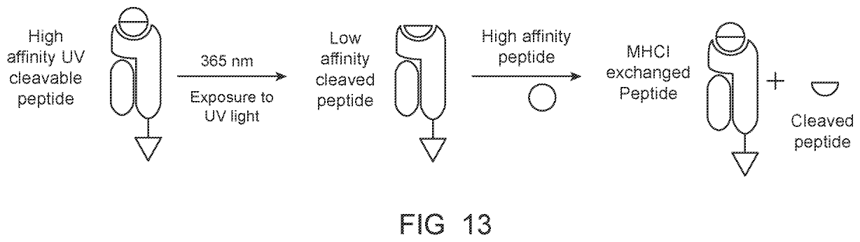

For UV-MHC class I complexes, the UV cleavable peptide was a conditional ligand that allows for an epitope peptide of interest to be exchanged into the peptide binding groove of the complex. The process is shown pictorially in FIG. 13.

UV-MHCs or peptide-MHC class I molecules are labeled with fluorophore. UV-MHC class I molecules are then loaded with a peptide of interest by UV mediated exchange to form the final probe.

Peptides. Synthetic peptides 8-11 amino acids in length, >90% purity, lyophilized were used. Peptides of interest were added at a molar excess to specific UV-MHCs for UV mediated exchange into the complex (see FIG. 13), or used to directly generate peptide-MHC class I molecules by refolding with B2M and HLA.

NHS-ester fluorophore. NHS-ester fluorophore was added in a molar excess to UV-MHC class I molecules or peptide-MHC class I molecules for conjugation to surface exposed lysine residues. Fluorophore selected must be able to withstand downstream exposure to UV light at 365 nm. An example of a fluorophore used was Alexa Fluor™ 488.

### Example 3: Probe Synthesis Method

Labeling of UV-MHC with fluorophore. UV-MHC of desired allele was incubated with a 2-20× molar excess of NHS-fluorophore in phosphate buffered saline pH 7.4 for 2 hours at room temperature. Excess, unconjugated fluorophore was removed by dialysis. The sample was loaded into a 10K molecular weight cutoff dialysis cassette (Slide-A-Lyzer™ 10K MWCO cassette, Thermo Fisher™) and placed in 25 mM TRIS pH 8.0, 150 mM NaCl, 4 mM EDA at a ratio of 1:2000, sample:dialysate. Sample and dialysate were incubated at 4° C. for 8 hours while continually mixing using a magnetic stir plate. Dialysate was then discarded and replaced for an additional 8-hour incubation at 4° C., while stirring. After the second round of dialysis, the sample was recovered, and protein concentration was determined using a UV-Visible spectrophotometer, and was corrected for the contribution of the fluorophore to the absorbance at 280 nm.

Determined degree of fluorophore labeling. Degree of fluorophore labeling was determined by reversed phase liquid chromatography mass spectrometry (RP LC-MS). 2-3 μg of the MHC-fluorophore conjugation reaction are injected on an Agilent™ 1290 Infinity™ series HPLC in line with an Agilent 6230™ time-of-flight electrospray ionization mass spectrometer. The sample was injected onto a reversed phase column (Agilent™ PLRP-S 1000 Å, 8 μm, 50×2.1 mm). The column was exposed to a gradient of 25-45% mobile phase B in 5 min at 0.50 mL/min with the column heated to 80° C. Mobile phase A was 0.05% TFA. Mobile phase B was 0.05% TFA in acetonitrile. The column eluent was sent to the LC-MS for mass spectrometry data acquisition. The degree of fluorophore conjugation can be determined by using the deconvoluted mass spectra of the peaks corresponding to B2M and HLA. The average number of fluorophores per pMHC class I molecule was calculated by using the relative abundance of each mass corresponding to a different number of fluorophore additions to each protein species and determining a weighted average for the complex.

UV mediated peptide exchange. Peptides were solubilized in ethylene glycol to a concentration of 20 mg/mL and added to fluorophore labeled peptide-MHC I molecule at a 25× molar excess. The peptide exchange reaction was performed in 25 mM TRIS pH 8.0, 150 mM NaCl, 4 mM EDTA, and contained 5% ethylene glycol v/v after the addition of peptide. The final concentration of the peptide-MHC I molecule in the exchange reaction was 2.0 mg/mL. The peptide-exchange reaction was performed in a UV transparent 96-well plate, or a clear, colorless sample tube, up to 15 mL in volume.

The peptide-exchange reaction was then incubated under a UV light set to 365 nm (Analytikjena™ UVP 3UV Lamp) for 20 minutes. The lamp was be placed as close as possible to the sample vessel. After exposure to UV light, the exchange reaction was allowed to proceed at room temperature for a minimum or 4 hours, or overnight incubation.

Determination of peptide binding to MHC. A 2-dimensional liquid chromatography mass spectrometry (2D LC-MS) method was used to characterize peptide binding to MHC class I complexes. Between 2-3 μg of MHC class I-peptide mixtures was injected on the instrument and sent to the first-dimension column. The first dimension LC method employed an analytical size exclusion column (SEC) (Agilent™ AdvanceBio™ SEC 300 Å, 2.7 um, 4.6×15 mm) to separate intact complex from excess peptide run at an isocratic flow of 0.7 ml/min in 25 mM TRIS pH 8.0, 150 mM NaCl for 10 min with signal acquisition at 280 nm. A sampling valve collects the entirety of the complex peak that eluted between 1.90-2.13 min in a volume of 160 μL and injects it onto the second dimension reversed phase column (Agilent™ PLRP-S™ 1000 Å, 8 um, 50×2.1 mm). The second-dimension column was exposed to a gradient of 5-50% mobile phase B in 4.7 min at 0.55 ml/min with the column heated to 80° C. Mobile phase A was 0.05% TFA. Mobile phase B was 0.05% TFA in acetonitrile. The column eluent was sent to an Agilent 6224 TOF LCMS for mass spectrometry data acquisition.

### Example 4: pMHC-OVA Association Assay

In a TCR association assay, OT-I cells were used which are originated from a murine transgenic line that produces MHC class I-restricted, ovalbumin-specific, CD8+ T cells (OT-I cells). The OT-I cells had a T cell antigen receptor that consists of α-chain variable region 2 (Vα2) and β-chain variable region 5 (Vβ5), which were inherited via a single transgene.

OT-I cells were first counted and had a viability of 82% with some dead cell debris. OT-I cells were resuspended at 4 million per mL in buffer (e.g., 1×PBS, 0.5% BSA+2 mM EDTA or 1×PBS, 0.5% BSA+0.05% sodium azide) and stained with Anti-TCRβ (H57-597 1/600) and a viability dye (1/1000). OT-I cells were then dispensed 100 μL into each well of a 96 well plate. OT-I cells were then washed by adding 150 μL of 1×PBS, 0.5% BSA+0.05% sodium azide buffer and spinning down at 1400 rpm for 2.5 minutes. Calibration beads were also stained as a way to measure the number of receptors at the cell surface.

Solutions including monomer probe complexes with ligand peptides, and IgG antibodies were prepared as follows:
1. N4 monomer 100 μg/mL=30 μL of 2 mg/mL monomer+570 μL of 1×PBS, 0.5% BSA+0.05% sodium azide buffer
2. N4 monomer 50 μg/mL=15 μL of 2 mg/mL monomer+585 μL of 1×PBS, 0.5% BSA+0.05% sodium azide buffer
3. N4 monomer 20 μg/mL=6 μL of 2 mg/mL monomer+594 μL of 1×PBS, 0.5% BSA+0.05% sodium azide buffer
4. T4 monomer 100 μg/mL=30 μL of 2 mg/mL monomer+570 μL of 1×PBS, 0.5% BSA+0.05% sodium azide buffer
5. T4 monomer 50 μg/mL=15 μL of 2 mg/mL monomer+585 μL of 1×PBS, 0.5% BSA+0.05% sodium azide buffer
6. T4 monomer 20 μg/mL=6 μL of 2 mg/mL monomer+594 μL of 1×PBS, 0.5% BSA+0.05% sodium azide buffer
7. V4 monomer 100 μg/mL=30 μL of 2 mg/mL monomer+570 μL of 1×PBS, 0.5% BSA+0.05% sodium azide buffer
8. V4 monomer 50 μg/mL=15 μL of 2 mg/mL monomer+585 μL of 1×PBS, 0.5% BSA+0.05% sodium azide buffer
9. V4 monomer 20 μg/mL=6 μL of 2 mg/mL monomer+594 μL of 1×PBS, 0.5% BSA+0.05% sodium azide buffer
10. IgG antibody (for staining beads) 100 μg/mL=33 μL of 1.8 mg/mL+557 μL of 1×PBS, 0.5% BSA+0.05% sodium azide buffer
11. peptide only 100 μg/mL=14.3 μL of 4.2 mg/mL+586 μL of 1×PBS, 0.5% BSA+0.05% sodium azide buffer

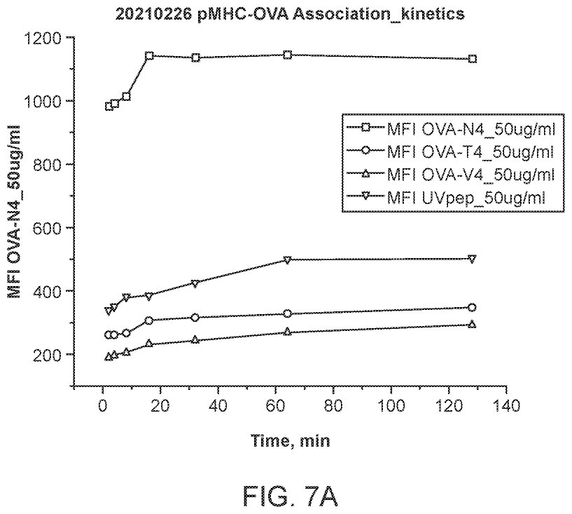

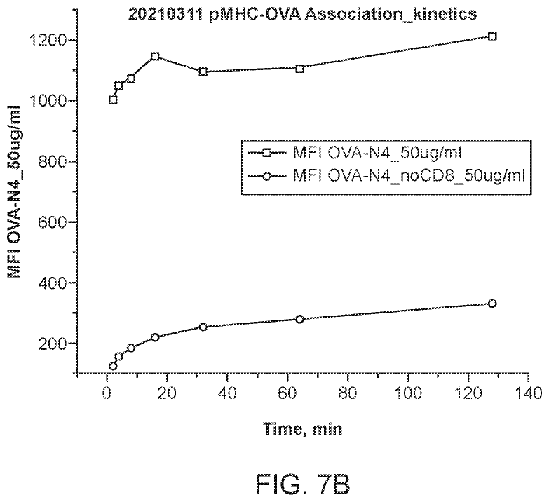

120 μL of solution with the monomers, peptides, and antibodies was added to a 96 well plate (a holding plate was for the reagents before transferring). A multichannel pipet was used to transfer 100 μL of each of the solutions to the cells. Cells were stained at timepoints 0, 3, 9, 27, and 81 minutes. At each time point, 150 μL of Cytofix™ buffer was added to the wells to stop the reaction and the cells were transferred to another plate. Cells were then incubated between 20 to 40 minutes at 4 degrees. Once the fixation was completed for each time point, the cells were spun down using a centrifuge operating at 1500 rpm for 2.5 min and then 200 μL of 1×PBS, 0.5% BSA+0.05% sodium azide buffer was added. All the cells were combined into one plate with the same template as staining for running in the flow cytometer. Cells were spun down using a centrifuge operating at 1500 rpm for 2.5 min and then 130 μL of buffer was added. 100 μL of the solution containing cells was run on a 1×PBS, 0.5% BSA+0.05% sodium azide at a rate of 0.5 μL per second (1 μL per second may be used). Data was collected and example data for N4 is shown graphically in FIGS. 7A and 7B.

To detect the level of phosphorylation of the ζ domain of TCR, cells were washed with 1× permeabilization buffer (Thermo Fisher Scientific™ 00-8333-56) prior to incubation with anti-pCD247 CD3ζ Tyr142 APC (Thermo Fisher Scientific™ 17-2478-42) diluted at 1:50 in 1× perm buffer for 30 minutes at room temperature. Cells were then washed with 1× permeabilization buffer and resuspended in buffer (e.g., 1×PBS, 0.5% BSA+2 mM EDTA or 1×PBS, 0.5% BSA+0.05% sodium azide) for subsequent flow cytometric analysis.

### Example 5: pMHC-OVA Dissociation Assay

In a TCR dissociation assay, OT-I cells were first counted and had a viability of 50%. A dead cell removal kit was used to remove dead cells. Cells were then counted again and had a viability of 75%.

400 k cells were then deposited into each well of a 96 well plate (not all the wells were used). Cells were spun down using a centrifuge and stained with anti-TCRβ (H57-597 1/600) and a viability dye (1/1000) in 1×PBS, 0.5% BSA+0.05% sodium azide buffer was added to each at 100 μL per well. The cells were incubated for 20 minutes at room temperature, then 150 μL of 1× PBS, 0.5% BSA+0.05% sodium azide buffer was added and cells were spun down using a centrifuge.

Solutions including monomer probe complexes with ligand peptides, and IgG antibodies were prepared as follows:
1. N4 monomer 100 μg/mL=30 μL of 2 mg/mL monomer+570 μL of 1×PBS, 0.5% BSA+0.05% sodium azide buffer
2. T4 monomer 100 μg/mL=30 μL of 2 mg/mL monomer+570 μL of 1×PBS, 0.5% BSA+0.05% sodium azide buffer
3. V4 monomer 100 μg/mL=30 μL of 2 mg/mL monomer+570 μL of 1×PBS, 0.5% BSA+0.05% sodium azide buffer
4. IgG antibody (for beads) 100 μg/mL=33 μL of 1.8 mg/mL+557 μL of 1×PBS, 0.5% BSA+0.05% sodium azide buffer
5. peptide only 100 μg/mL=14.3 μL of 4.2 mg/mL+586 μL of 1×PBS, 0.5% BSA+0.05% sodium azide buffer

The 100 μL samples were then transferred to a plate containing cells using a multi-channel pipette to minimize time difference. The cells were then incubated at room temperature for one hour. Samples were topped with buffer and spun down using a centrifuge. Two more washes were completed using 200 μL of 1×PBS, 0.5% BSA+0.05% sodium azide buffer.

Samples were then incubated at room temperature for time points 0, 3, 9, 27, and 81 minutes. At the time points, 300 μL of Cytofix™ buffer was added and mixed with each sample. The samples were then transferred to a new plate and fixed for 20-40 minutes, spun down using a centrifuge, and resuspended in (e.g., 1×PBS, 0.5% BSA+2 mM EDTA or 1×PBS, 0.5% BSA+0.05% sodium azide) buffer.

To detect the level of phosphorylation of the ζ domain of TCR, cells were washed with 1×permeabilization buffer (Thermo Fisher Scientific™ 00-8333-56) prior to incubation with anti-pCD247 CD3ζ Tyr142 APC (Thermo Fisher Scientific™ 17-2478-42) diluted at 1:50 in 1× permeabilization buffer for 30 minutes at room temperature. Cells were then washed with 1× permeabilization buffer and resuspended in (e.g., 1×PBS, 0.5% BSA+2 mM EDTA or 1×PBS, 0.5% BSA+0.05% sodium azide) buffer for subsequent flow cytometric analysis.

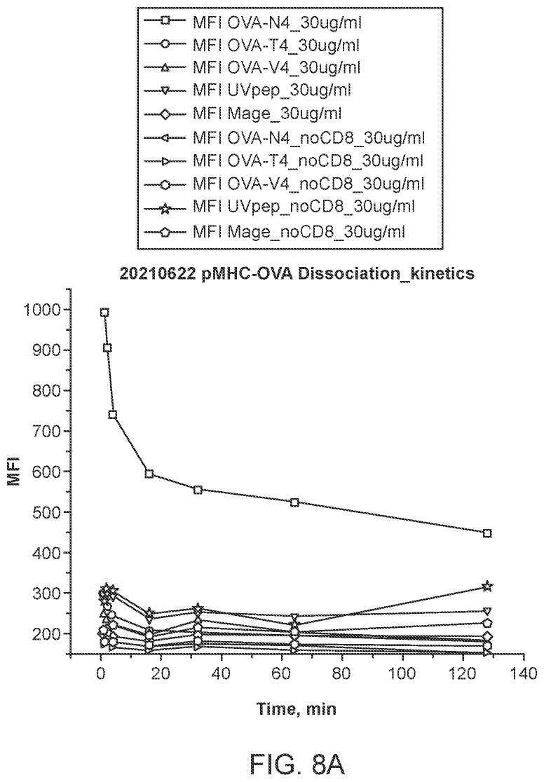

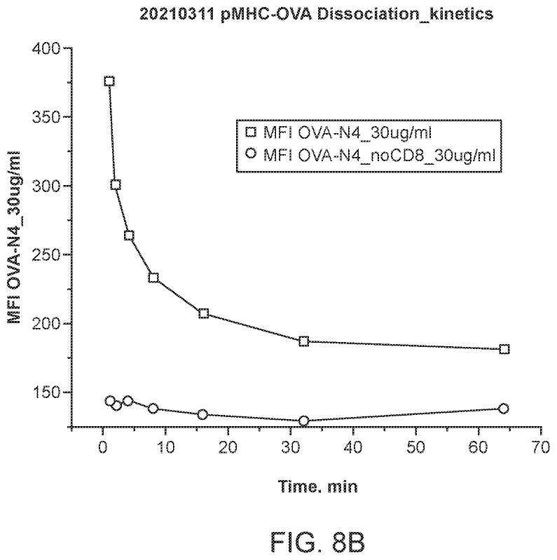

Cells were then spun down using a centrifuge and resuspend in 130 μL of 1×PBS, 0.5% BSA+0.05% sodium azide buffer. Samples were then run on a BD Symphony™ using a plate reader at 0.5 μL per second. Data was collected and example data for N4 is shown graphically in FIGS. 8A and 8B.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification can have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences can be varied and still remain within the spirit and scope of the various embodiments.

1. A composition for use in a biokinetic assay, the composition comprising:
a polypeptide molecule comprising an antigen-binding region;
one or more fluorophores linked to the polypeptide molecule; and
an antigenic peptide bound to the antigen-binding region of the polypeptide molecule.

2. A composition for use in a biokinetic assay, the composition comprising:
a polypeptide molecule comprising an antigen-binding region;
one or more fluorophores linked to the polypeptide molecule; and
an antigenic peptide bound to the antigen-binding region of the polypeptide molecule, wherein the antigenic peptide comprises a UV cleavable amino acid.

3. The composition according to any one of the preceding claims, wherein the polypeptide molecule comprises a single polypeptide chain.

4. The composition of claim 3, wherein the polypeptide molecule comprises a first polypeptide chain and a second polypeptide chain.

5. The composition of claim 4, wherein the antigen-binding region comprises at least a portion of the first polypeptide chain of the polypeptide molecule.

6. The composition of claim 5, wherein the first polypeptide chain comprises an α₁ domain and an α₂ domain.

7. The composition of claim 6, wherein the first polypeptide chain comprises an α₃ domain.

8. The composition according to any one of claims 3-7, wherein the first polypeptide chain comprises an amino acid sequence of a recombinant human leukocyte antigen.

9. The composition according to any one of claims 3-7, wherein the first polypeptide chain comprises an amino acid sequence of a recombinant murine histocompatibility system 2.

10. The composition according to any one of claims 6-9, wherein the second polypeptide chain is linked to the α₃ domain of the first polypeptide chain.

11. The composition according to any one of claims 3-10, wherein the second polypeptide chain comprises an amino acid sequence of a β₂-microglobulin molecule.

12. The composition of claim 3, wherein the antigen-binding region comprises at least a portion of the first polypeptide chain and at least a portion of the second polypeptide chain.

13. The composition of claim 12, wherein the portion of the first polypeptide chain comprises an α₁ domain, the portion of the second polypeptide chain comprises a β₁ domain, and the α₁ domain and the β₁ domain form the antigen-binding region.

14. The composition of claim 13, wherein the first polypeptide chain further comprises an α₂ domain.

15. The composition of claim 13, wherein the second polypeptide chain further comprises a β₂ domain.

16. The composition according to any one of claims 12-15, wherein the first polypeptide chain comprises an amino acid sequence of a recombinant human leukocyte antigen.

17. The composition according to any one of claims 12-16, wherein the second polypeptide chain comprises a polypeptide sequence of a recombinant human leukocyte antigen.

18. The composition of claim 17, wherein the antigen-binding region comprises at least one α-helix.

19. The composition according to any one of claims 17-18, wherein the antigen-binding region comprises at least one β-sheet.

20. The composition according to any one of the preceding claims, wherein the one or more fluorophores are covalently linked to the polypeptide molecule.

21. The composition of claim 20, wherein the covalent linkage comprises an ester.

22. The composition according to any one of the preceding claims, wherein the one or more fluorophores are covalently linked to one or more solvent exposed surface lysine residues of the polypeptide molecule.

23. The composition according to any one of claims 3-22, wherein the first and second polypeptide chains are non-covalently linked.

24. The composition according to any one of claims 3-22, wherein the first and second polypeptide chains are covalently linked.

25. The composition according to any one of the preceding claims, wherein the polypeptide molecule is bound to an antigen presenting cell surrogate.

26. The composition of claim 25, wherein the antigen presenting cell surrogate comprises a bead.

27. The composition according to any one of claims 25-26, wherein the polypeptide molecule is attached to the antigen presenting cell surrogate by a linker.

28. The composition of claim 27, wherein the linker comprises a polyethylene glycol (PEG) molecule.

29. The composition according to any one of the preceding claims, further comprising a receptor bound to the antigen-binding region of the polypeptide molecule.

30. The composition of claim 29, further comprising a live lymphocyte, wherein at least a portion of the receptor is on a cell membrane of the live lymphocyte.

31. The composition of claim 30, further comprising a co-receptor on the cell membrane of the live lymphocyte, wherein the co-receptor is bound to a portion of the polypeptide molecule.

32. The composition of claim 31, wherein the live lymphocyte is a T cell and the receptor is a T cell receptor (TCR).

33. The composition of claim 32, wherein the co-receptor comprises a CD8 molecule.

34. The composition of claim 32, wherein the co-receptor comprises a CD4 molecule.

35. The composition of claim 31, wherein the live lymphocyte is a B cell and the receptor is a B cell receptor (BCR).

36. The composition of claim 31, wherein the live cell comprises a macrophage and the receptor comprises a chemokine receptor.

37. The composition of claim 31, wherein the live cell comprises a dendritic cell and the receptor comprises a pattern recognition receptor (PRR).

38. The composition according to any one of the preceding claims, wherein the antigenic peptide has a length that ranges from 8-11 amino acid residues.

39. The composition according to any one of claims 1-37, wherein the antigenic peptide has a length that ranges from 15-24 residues.

40. The composition according to any one of the preceding claims, wherein the antigenic peptide comprises a UV moiety.

41. A reaction mixture for generating a probe complex for use in a biokinetic assay, the mixture comprising:
the composition of claim 40; and
a target antigenic peptide.

42. The reaction mixture of claim 41, wherein the target antigenic peptide is present at a concentration in a molar excess compared to the antigenic peptide bound to the antigen-binding region of the polypeptide molecule.

43. The reaction mixture according to any one of claims 41-42, wherein the concentration of the target antigenic peptide is 25 times greater than the concentration of the antigenic peptide.

44. The reaction mixture according to any one of claims 41-43, wherein the reaction mixture comprises 25 mM TRIS.

45. The reaction mixture according to any one of claims 41-44, wherein the reaction mixture is at a pH of 8.0.

46. The reaction mixture according to any one of claims 41-45, wherein the reaction mixture comprises 150 mM NaCl.

47. The reaction mixture according to any one of claims 41-46, wherein the reaction mixture comprises 4 mM EDTA.

48. The reaction mixture according to any one of claims 41-47, wherein the reaction mixture comprises 5% ethylene glycol.

49. A method of generating a monomeric probe complex, comprising:
contacting a polypeptide molecule comprising an antigen binding region with a first antigenic peptide to generate an antigen-presenting complex;
contacting the antigen-presenting complex with a plurality of fluorophore molecules to generate a fluorophore-labeled antigen-presenting complex;
determining a quantity of the fluorophore molecules covalently bound to the fluorophore-labeled antigen-presenting complex; and
exchanging the first antigenic peptide with a second antigenic peptide to generate the monomeric probe complex.

50. The method of claim 49, wherein exchanging the first antigenic peptide with the second antigenic peptide comprises cleaving the first antigenic peptide to generate a cleaved first antigenic peptide, wherein the cleaved first antigenic peptide has a lower binding affinity for the antigen binding region than the first antigenic peptide.

51. The method of claim 50, wherein cleaving the first antigenic peptide comprises applying UV radiation.

52. The method of claim 50, wherein the second antigenic peptide has a higher affinity for the antigen binding region than the cleaved first antigenic peptide.

53. The method of claim 51, wherein the UV radiation comprises a wavelength of 365 nanometers.

54. The method according to any one of claims 49-53, wherein the step of contacting the antigen-presenting complex with a plurality of fluorophore molecules to generate a fluorophore-labeled antigen-presenting complex comprises covalently linking one or more solvent exposed surface lysine residues of the polypeptide molecule to one or more of the fluorophore molecules.

55. The method according to any one of claims 49-54, further comprising separating one or more unconjugated fluorophores from the labeled monomeric probe complex.

56. The method of claim 55, further comprising determining a quantity of a plurality of labeled monomeric probe complexes.

57. The method of claim 56, further comprising determining an average number of fluorophores conjugated to each of a plurality of the labeled monomeric probe complexes.

58. The method of claim 57, wherein the step of determining the average number of fluorophores conjugated to each of the plurality of the labeled monomeric probe complexes includes using a plurality of relative abundance values, wherein each relative abundance value corresponds to a different number of conjugated fluorophores.

59. The method of claim 58, wherein each of the plurality of relative abundance values are determined using mass spectrometry.

60. A method for collecting association rate data for assessing live cell activation, comprising:
contacting a plurality of live cells with a plurality of compositions of any one of claims 1-29 at a concentration, wherein each of the plurality of live cells comprises a plurality receptor molecules on a cell membrane;
binding the receptor molecules to the compositions over a time interval to form a plurality of receptor-probe complexes, wherein each receptor-probe complex comprises one composition bound to one receptor;
collecting at least two samples of the plurality of live cells at different time points over the time interval;
contacting the live cells in each of the at least two samples with a fixing agent to preserve the receptor-probe complexes and to prevent further binding between the receptor molecules and the compositions;
determining a number of receptor-probe complexes on the cell membrane of each cell; and
analyzing the number of receptor-probe complexes on the cell membrane of each cell in each sample to collect the association rate data.

61. A method for collecting dissociation rate data for assessing live cell activation, comprising:
contacting a plurality of live cells with a plurality of compositions of any one of claims 1-29 at a concentration, wherein each of the plurality of live cells comprises a plurality receptor molecules on a cell membrane;
binding the receptor molecules to the compositions to form a plurality of receptor-probe complexes until an equilibrium is achieve, wherein each receptor-probe complex comprises one composition bound to one receptor;
dissociating a portion of the receptor-probe complexes over a time interval by reducing the concentration of the compositions;
collecting at least two samples of the plurality of live cells at different time points over the time interval;
contacting the live cells in each of the at least two samples with a fixing agent to preserve the receptor-probe complexes and to prevent further binding between the receptor molecules and the compositions;
determining a number of receptor-probe complexes on the cell membrane of each cell; and
analyzing the number of receptor-probe complexes on the cell membrane of each cell in each sample to collect the association rate data.

62. The method according to any one of claims 60-61, wherein the live cells comprise a T cell.

63. The method according to any one of claims 60-61, wherein the live cells comprise a B cell.

64. The method according to any one of claims 60-61, wherein the live cells comprise a macrophage.

65. The method according to any one of claims 60-61, wherein the live cells comprise a dendritic cell.

66. The method according to any one of claims 60-65, wherein the signal intensity of the composition is measured using an analytical device.

67. The method of claim 66, wherein the analytical device comprises a flow cytometer.

68. The method of claim 66, wherein the analytical device comprises a fluorometer.

69. The method according to any one of claims 60-68, wherein the fixing agent comprises paraformaldehyde.

70. The method according to any one of claims 60-69, further comprising preventing receptor internalization by decreasing a temperature of the receptor-probe complexes.

71. The method of claim 70, wherein the temperature is decreased to 4° C.

72. The method according to any one of claims 60-71, further comprising separating one or more unbound compositions from the plurality of live cells.

73. The method according to any one of claims 60-72, wherein each of the receptor-probe complexes comprises a CD8 molecule.

74. The method according to any one of claims 60-72, wherein each of the receptor-probe complexes comprises a CD4 molecule.

75. The method according to any one of claims 60-74, further comprising permeabilizing the cell membrane of each cell.

76. The method of claim 75, further comprising applying a detection reagent for detecting a level of phosphorylation of an intracellular domain of each of the live cells.

77. The method of claim 76, wherein the intracellular domain comprises a ζ domain.

78. The method of claim 77, wherein the detection reagent comprises an antibody.

79. A system for measuring binding kinetics for assessing live cell activation, comprising:
an analytical device comprising an analysis chamber, wherein the analysis chamber comprises a composition according to any one of claims 1-40.

80. A system for measuring binding kinetics for assessing live cell activation, comprising:
an analytical device comprising an analysis chamber, wherein the analysis chamber comprises a reaction mixture according to any one of claims 41-48.

81. The system according to any one of claims 79-80, wherein the analysis device comprises:
an optical source for interrogating the analysis chamber; and
a detector for detecting a signal.

82. The system of claim 81, further comprising a computer system comprising a non-transitory computer readable storage medium, wherein the non-transitory computer readable storage medium comprises instructions for analyzing the signal.

83. The system of claim 82, wherein analyzing the signal comprises normalizing the signal to a previously determining calibration value.

84. The system according to any one of claims 79-83, wherein the analysis chamber comprises a flow cell.

85. The system according to any one of claims 80-84, wherein the analytical device comprises a flow cytometer.

86. The system according to any one of claims 80-84, wherein the analytical device comprises a fluorometer.

87. The system according to any one of claims 80-84, wherein the analytical device comprises a microscope.

88. The system according to any one of claims 80-84, wherein the analytical device comprises a mass spectrometer.

89. A method for collecting biophysical parameter data for assessing a T cell receptor (TCR) and a peptide-major histocompatibility complex (MHC) association rate constant, comprising:
generating a set of monomeric probes, wherein each monomeric probe comprises a detection molecule and one MHC comprising a peptide;
associating TCR molecules with the monomeric probes in a one-to-one correspondence to form TCR-monomeric-probe complexes over a time interval;
sampling two or more subsets of TCR-monomeric-probe complexes over the time interval, wherein each subset is taken at a different timepoint;
preventing formation of new TCR-monomeric-probe complexes within each subset at their corresponding timepoints; and
measuring a signal intensity from the detection molecules in each subset using an analytical device.

90. The method of claim 89, wherein the TCR molecule is expressed on a T cell.

91. The method according to any one of claims 89-90, wherein the detection molecule comprises a fluorescent molecule.

92. The method according to any one of claims 89-91, wherein detection molecule and MHC are coupled with a linker.

93. The method of claim 92, wherein the linker comprises a biotinylated structure.

94. The method of claim 92, wherein the linker comprises PEG

95. The method according to any one of claims 89-94, wherein the analytical device comprises a flow cytometer.

96. The method according to any one of claims 89-95, wherein the step of preventing comprises use of a fixation buffer.

97. The method according to any one of claims 89-96, further comprising decreasing a temperature following the preventing step.

98. The method of claim 97, wherein the temperature is 4 degrees C.

99. The method according to any one of claims 89-98, further comprising separating unbound monomeric probes from the TCR molecules.

100. The method according to any one of claims 89-99, wherein the time interval begins prior to an equilibrium state of TCR-monomeric-probe complexes.

101. The method according to any one of claims 89-100, wherein the monomeric probe further comprises a CD8 complex.

102. A method for collecting biophysical parameter data for assessing a T cell receptor (TCR) and a peptide-major histocompatibility complex (pMHC) dissociation rate constant, comprising:
generating a set of monomeric probes, wherein each monomeric probe comprises a detection molecule and one MHC comprising a peptide;
associating TCR molecules with the monomeric probes in a one-to-one correspondence to form TCR-monomeric-probe complexes;
dissociating the TCR-monomeric-probe complexes into monomeric probes and TCRs over a time interval;
sampling two or more subsets of TCR-monomeric-probe complexes over the time interval, wherein each subset is taken at a different timepoint;
preventing dissociation of additional TCR-monomeric-probe complexes within each subset at their corresponding timepoints; and
measuring a signal intensity from the detection molecules in each subset using a high-throughput analytical device.

103. The method of claim 102, wherein the TCR molecule is expressed on a T cell.

104. The method according to any one of claims 102-103, wherein the detection molecule comprises a fluorescent molecule.

105. The method according to any one of claims 102-104, wherein detection molecule and MHC are coupled with a linker.

106. The method of claim 105, wherein the linker comprises a biotinylated structure.

107. The method of claim 105, wherein the linker comprises PEG

108. The method according to any one of claims 102-107, wherein the analytical device comprises a flow cytometer.

109. The method according to any one of claims 102-108, wherein the step of preventing comprises use of a fixation buffer.

110. The method according to any one of claims 102-109, further comprising decreasing a temperature following the preventing step.

111. The method of claim 110, wherein the temperature is 4 degrees C.

112. The method according to any one of claims 102-111, further comprising separating unbound monomeric probes from the TCR molecules.

113. The method according to any one of claims 102-112, wherein the time interval begins at an equilibrium state of TCR-monomeric-probe complexes.

114. The method according to any one of claims 102-113, wherein the dissociation step uses a buffer agent to dilute a solution comprising the TCR-monomeric-probe complexes.

115. The method according to any one of claims 102-114, wherein the monomeric probe further comprises a CD8 complex.
